(19) United States
(12) Patent Application Publication
(10) Pub. No.: US 2014/0033367 A1
(43) Pub. Date: Jan. 30, 2014

(54) AGRONOMIC TRAITS VIA GENETICALLY INDUCED ELEVATION OF PHYTOHORMONE LEVELS IN PLANTS

(71) Applicant: Henry Daniell, Winter Park, FL (US)

(72) Inventor: Henry Daniell, Winter Park, FL (US)

(73) Assignee: University of Central Florida Research Foundation, Orlando, FL (US)

(21) Appl. No.: 13/809,188

(22) PCT Filed: Jul. 11, 2011
(86) PCT No.: PCT/US2011/043565

§ 371 Date: Jun. 10, 2013

Related U.S. Application Data
(60) Provisional application No. 61/363,188, filed on Jul. 9, 2010.

(51) Int. Cl. C12N 15/82

(52) U.S. Cl. CPC C12N 15/8261
USPC 800/290; 800/298; 800/320.1; 800/320.2; 800/320; 800/320.3; 800/312; 800/317.2; 800/317.3; 800/306; 800/317.4; 800/314; 800/313; 800/315; 800/309; 800/317; 800/317.1; 435/468

(57) ABSTRACT

Disclosed herein are transgenic plants that have been modified to express β-glucosidase, and methods and materials for making same. The transgenic plants possess several advantageous features including increased biomass, increased trichome density and increased parasite resistance.

### RELATED APPLICATIONS

This application claims priority to U.S. Ser. No. 61/363,188, filed Jul. 9, 2010, which is incorporated herein in its entirety.

### GOVERNMENT SUPPORT

This work was supported by NIH R01 GM 63879 and USDA 3611-21000-021-02S grants. The U.S. government has certain rights in this invention.

### BACKGROUND

In plants, β-glucosidases have been implicated in key developmental processes, such as growth, pathogen defense, and hormone hydrolysis (Esen, 1993; Kleczkowski and Schell, 1995). Recently, considerable progress has been made in elucidating the functions of β-glucosidases for chemical plant defense against pathogens (Morant et al., 2008) and activation of plant hormone groups, including auxins, abscisic acid (ABA), and cytokinins (Wiese and Grambow, 1986; Brzobohatý et al., 1993; Lee et al., 2006). Fungal β-glucosidases efficiently hydrolyze GA-13-O-glucosides. In contrast, enzyme from plants exhibit very low activity (Schliemann and Schneider, 1979; Schliemann, 1984; Sembdner et al., 1994). However, very little is known about the contribution of β-glucosidases to GA homeostasis (Schneider and Schliemann, 1994).

Hormones play an important role in regulating plant growth and development (Davies, 2004). Their regulating properties appear in the course of the biosynthetic and signaling pathways and are followed by catabolic processes. All these metabolic steps are irreversible except for some processes including the formation of glucoside ester or ether conjugates, where the free hormone can be liberated by β-glucosidase enzymatic hydrolysis. For each class of the plant hormones, conjugates have been found (Kleczkowski and Schell, 1995). After characterization of the first GA glucoside, GA₈-2-O-β-d-glucoside from Phaseolus coccineus fruits (Schreiber et al., 1970), the term GA conjugate was used for a GA covalently bound to another low-molecular-weight compound. There is now evidence that hormone conjugates act as reversible deactivated storage forms and are important in the regulation of physiologically active hormone levels (Schneider and Schliemann, 1994). The conjugation process is an important aspect of hormone metabolism in plants but has not yet been explored in enhancing growth or productivity.

The most common GA conjugates isolated from plants are connected to Glc. These conjugates are divided into two groups: glucosyl ethers (or O-glucosides) and glucosyl esters. GA Glc conjugates are biologically inactive. The degree of hydrolysis reflects the activity of released parent GA (Sembdner et al., 1980). The loss of biological activity in the course of the conjugation process and the increased polarity of GA glucosyl conjugates favor GA conjugates for their deposition into the plant cell vacuole, but their storage within chloroplasts has not yet been investigated. Because of their preferential formation and accumulation during seed maturation, it has been proposed that GA Glc conjugates may function as storage products (Schneider et al., 1992). The easy formation and hydrolysis of GA glucosyl conjugates results in reversible deactivation/activation and facilitates the regulation of free GA pools.

Plastids play an important role in early biosynthetic steps of plant hormones, including auxins, cytokinins, ABA, and GAs (Davies, 2004). Proplastids of the apical meristem are reported to contain enzymes involved in the early biosynthesis of GAs, but their activities are not detected in mature chloroplasts (Aach et al., 1997; Yamaguchi et al., 2001). Although cytokinins affect a number of processes in chloroplasts, their metabolism has not been fully understood. Chloroplasts from dark-treated tobacco (Nicotiana tabacum) leaves were reported to contain zeatin riboside-O-glucoside and dihydrozeatin riboside-O-glucoside and relatively high cytokinin oxidase activity, suggesting that chloroplasts may contain cytokinins, their conjugates, and the enzymatic activity necessary for their metabolism (Benková et al., 1999). However, a similar role of plastids for subcellular hormone homeostasis is not known for GAs, auxins, and ABA (Nambara and Marion-Poll, 2005; Woodward and Bartel, 2005; Marion-Poll and Leung, 2006).

Sap-sucking insects belonging to the order Homoptera include some of the most devastating insect pests worldwide. Most serious damage caused by these pests is due to their role as vectors of plant viruses. Morphologically specialized structures such as trichomes located on the plant surface may serve as physical barriers to prevent insect feeding. It is well known that trichomes secrete secondary metabolites that are toxic to insects. Among these metabolites, Suc esters are predominant and highly toxic to whiteflies (Bemisia tabaci; Severson et al., 1984; Lin and Wagner, 1994). Cembrenoid diterpene has neurotoxic, cytotoxic, and antimitotic activities (Guo and Wagner, 1995).

### SUMMARY

Embodiments of the invention are based on research involving the development of transgenic plants (e.g. transformation of the nuclear or plastid genome) expressing a heterologous β-glucosidase gene (e.g. Bgl gene), which is believed to release plant hormones from their conjugates. Also, the affect of this expression plant development has been studied. It has been demonstrated that the transplastomic lines show early flowering and increases in biomass, height, internode length, leaf area, and density of leaf globular trichomes that contain more sugar esters that confer protection from whitefly and aphid (Myzus persicae) attacks. Many of the observed effects are typical for plants with altered GA levels (Pimenta Lange and Lange, 2006). In addition trans-zeatin, indole-3-acetic acid (IAA), and ABA levels were evaluated in different plant tissues or organs. The studies disclosed herein enable the modification of plants for enhancing biomass and conferring novel, advantageous plant traits.

### BRIEF DESCRIPTION OF THE DRAWINGS

FIG. 1. Chloroplast vectors, plant transformation, and transgene integration. A and B, Schematic representations of the chloroplast flanking sequences used for homologous recombination, probe DNA sequence (0.81 kb; A), and primer annealing sites (3P/3M, 5P/2M; B). C, First round of selection and primary transplastomic shoots. D, Second round of selection. E, Regenerated shoots on rooting medium for the third round of selection; all selection media contained spectinomycin (500 mg L⁻¹). F and G, PCR analysis using primer pairs 3P/3M and 5P/2M for evaluation of site-specific integration of the transgene cassette into the chloroplast genome. M, One-kilobase plus DNA ladder; P, positive control; T1 to T4, transplastomic lines; W, wild-type control. H, Southern blot hybridized with the flanking sequence probe. The UT chloroplast genome shows a 4.0-kb fragment, while BGL-1 lines show a 7.8-kb hybridizing fragment.

FIG. 2. Evaluation of transgene segregation and the phenotype of transplastomic (BGL-1) and UT plants. A, UT and transplastomic seeds germinated on half-strength MS medium containing spectinomycin (500 mg L⁻¹) confirm the lack of Mendelian segregation. B, Plants at 3 weeks after seed germination: UT (left) and BGL-1(right). C, Two-month-old transplastomic (left) and UT (right) plants. D, Leaves of transplastomic (bottom row) and UT (top row) plants. E, Mature (3-month-old) transplastomic (right) and UT (left) plants.

FIG. 3. Evaluation of leaf surface by scanning electron microscopy. A, Trichomes on leaf upper surface of a UT plant. B, Trichomes on leaf upper surface of a BGL-1 plant. C, Trichomes on leaf lower surface of a UT plant. D, Trichomes on leaf lower surface of a BGL-1 plant.

FIG. 4. Endogenous ABA, IAA, and trans-zeatin concentration in BGL-1 and UT plants. A, The trans-zeatin concentrations of BGL-1 and UT plants. B, The IAA concentrations of BGL-1 and UT plants. C, The ABA concentrations of BGL-1 and UT plants. ABA, IAA, and trans-zeatin concentrations were calculated as ng per g fresh weight (FW). Each measurement was replicated three to four times using different pooled samples and the Phytodetek competitive ELISA kit.

FIG. 5. Protoplasts and protoplast-derived cells and cell colonies. A, Protoplasts from UT leaf could not divide in the medium without hormones. B, First division of BGL-1 protoplast without hormones. C, Protoplasts from UT leaf could not divide in the medium with zeatin-O-glucoside. D, First cell division of BGL-1 sample in the medium with zeatin-O-glucoside. E, Protoplasts from UT leaf could not form calli in the medium without hormones. F, Protoplast-derived calli of BGL-1 sample in the medium without hormones. Bars=60 μm.

FIG. 6. Histochemical staining of sugar ester. A: Glandular trichomes stained by rhodamine B. B: Density of trichomes with red glandular heads stained by rhodamine B. C: Aphids from a UT plant showing lower staining intensity. D: Red aphids walking on the surface of a BGL-1 leaf. E: Glandular trichomes stained by rhodamine B from a UT plant: F: Higher density of red glandular trichomes from a BGL-1 leaf.

FIG. 7 Aphid and whitefly bioassays on BGL-1 and UT plants. A, The mesh-bag cage placed on each pot (40-d-old plants, six- to seven-leaf stage) on day 0 for insect bioassays. B, Plants 25 d after insect bioassays. C, Release of plants from the cage at 25 d after insect bioassays. D and E, A UT plant heavily colonized with mature and immature whiteflies. E shows an enlarged view of the circled area in D. F, BGL-1 transplastomic plants with negligible colonization of whiteflies. G and H, A UT plant heavily colonized with mature and immature aphids. H shows an enlarged view of the circled area in G. I, BGL-1 transplastomic plants with negligible aphids.

FIG. 8. Toxicity LD₅₀ values for whiteflies and aphids from trichome exudates of UT and BGL-1 plants. Analysis is of composite data from four independent experiments with separate exudate isolates. Mortality was assessed after 66 h. LD₅₀ values were estimated according to the Karber method.

FIG. 9. Schematic representation of pCR BluntII Topo vector containing different vacuolar targeting sequences fused to bgl1 coding sequence with or without his tag and pCAMBIA 2300 S vector.

FIG. 10. Selection of tobacco transgenic plants and confirmation of Bgl1 gene integration by PCA. A—Putative transgenic lines growing in regeneration medium containing kanamycin, B&C—Transgenic lines on rooting medium containing kanamycin; D—PCR analysis using Bgl1 gene specific primers (Lanes 1-13: different transgenic lines; UT: untransformed plants.

FIG. 11. Selection of Artemisia transgenic lines and confirmation of Bgl1 gene integration by PCT. A & B—Putative Bgl1 transformants growing on selection medium; C—Bgl1 transgenic lines on rooting medium containing kanamycin; D—PCR analysis using Bgl1 gene specific primers (M: Marker; Lanes 1-9: Bgl1 transgenic lines; Lane 10: Untransformed).

FIG. 12. Confirmation of Bgl1 transcript by Northern Blot using Bgl1 probe. A: Tobacco (UT: Untransformed plant; Lanes 1-8: Transgenic lines), B: Artemisia (Lanes 1-9: Transgenic lines, UT: Untransformed plant).

FIG. 13. T0 Bgl1 transgenic lines growing in green house. A: Tobacco, B: Artemisia (UT: Untransformed & T: Transgenic plants).

FIG. 14. Bgl1 enzyme activity assay of tobacco transgenic line using pNPG substrate at different pH. Transgenic line showed 4 folds enzyme activity than untransformed plant.

FIG. 15. Segregation of T0 Bgl1 tobacco seeds. A to D: different transgenic lines germinated on kanamycin containing germination medium. E: Different T1 Bgl1 transgenic plants growing in green house; F: Phenotypic comparison of untransformed and Bgl1 transgenic line (U: Untransformed plant, T: Transgenic line).

FIG. 16. SEM of Artemisia untransformed and Bgl1 transgenic lines showing trichomes.

FIG. 17. Putative transformant shoots for different transgenic lines of lettuce growing on selection medium.

FIG. 18. Confirmation of transgene integration by PCR analysis using nptII specific primers (UT: untransformed).

FIG. 19. Southern blot analysis of transgenic lines to determine transgene integration and copy number.

FIG. 20. Northern blot analysis of transgenic lines to determine presence of Bgl1 transcript.

FIG. 21. Gel diffusion assay to determine β glucosidase activity using fluorescent substrate 4-MUG. (Top row—commercial enzyme standard, middle row—wild type plant crude extract, bottom row—transgenic plant crude extract).

FIG. 22. Enzyme activity assay using pNPG as substrate. FIG. 6A—assay with 100 μg of wild type crude extract, FIG. 6B—assay with 100 μg transgenic crude extract.

FIG. 23. Optimization of pH. The activity is expressed based on release of the product p-nitrophenol (PNP). Blue line—wild type, Red line—transgenic.

FIG. 24. Optimization of enzyme reaction temperature. Blue line—wild type, red line—transgenic.

FIG. 25. Optimization of substrate concentration. Blue line—wild type, Red line—transgenic.

### DETAILED DESCRIPTION

It has been discovered that plants can be engineered to release native phytohormones stored in cellular compartments. Accordingly, in one aspect, the present invention includes a transgenic plant that displays an altered phenotype relative to the wild-type plant. In another embodiment, the transgenic plant has altered β-glucosidase expression.

According to another embodiment, the invention pertains to a method of producing a transgenic plant with Bgl overexpression relative to a wild-type plant. The method involves (a) introducing into a plant cell an expression cassette that includes a Bgl gene to thereby produce a transformed plant cell; and (b) producing a transgenic plant from the transformed plant cell. The resulting transgenic plant has increased biomass, increased height, increased trichome density or increased seed production relative to a wild type plant.

According to another embodiment, the invention pertains to a stably transformed plastid of a target plant. The plastid is transformed with a plastid transformation vector that includes an expression cassette having, as operably linked components a promoter operative in said plastid, a selectable marker sequence, a heterologous polynucleotide sequence coding for Bgl gene, transcription terminator functional in said plastid, and flanking each side of the expression cassette, flanking DNA sequences which are homologous to a DNA sequence of a plastid genome of said plastid. The stable integration of the heterologous coding sequence into the plastid genome of the target plant is facilitated through homologous recombination of the flanking sequence with the homologous sequences in said plastid genome.

In yet a further embodiment, the invention pertains to a stably transformed plant cell of a target plant. The plant cell is transformed with a transformation vector that includes an expression cassette that includes, as operably linked components, a promoter operative, a selectable marker sequence, a heterologous polynucleotide sequence coding for Bgl gene, a terminator, and flanking each side of the expression cassette, flanking DNA sequences which are homologous to a DNA sequence of a nuclear or plastid genome of said plant cell. The stable integration of the heterologous coding sequence into the nuclear or plastid genome of the target plant is facilitated through homologous recombination of the flanking sequence with the homologous sequences in said nuclear or plastid genome.

A further embodiment is directed to a method of producing a transgenic plant having increased trichome density. The method involves introducing into a plant cell an expression cassette that comprises a Bgl gene linked to a vacuole targeting sequence to thereby produce a transformed plant cell; and (b) producing a transgenic plant from the transformed plant cell, wherein the transgenic plant has increased trichome density.

According to an additional embodiment, the invention pertains to a method of releasing native phytohormones associated with a plant cell. The method involves engineering the plant cell so as to express heterologous Bgl1, wherein expression of the heterologous Bgl1 increases B-glucosidase activity in the cell which releases native phytohormones in said plant cell. In a specific embodiment, the native phytohormones are in a conjugated state prior to being exposed to B-glucosidase expressed in said plant cell. In a more specific embodiment, the native phytohormones are present in a vacuole of said plant cell. In an even more specific embodiment, the exposure of the native phytohormones to B-glucosidase occurs in the vacuole.

In yet another embodiment, the invention pertains to a method of producing a transgenic plant having increased parasite resistance. The method involves introducing into a plant cell an expression cassette that comprises a Bgl gene linked to a vacuole targeting sequence to thereby produce a transformed plant cell; and (b) producing a transgenic plant from the transformed plant cell, wherein the transgenic plant has increased plant parasite resistance. The plant has increased resistance to insect intrusion as compared to a wild-type of the same plant.

Any of the polynucleotides or polypeptides described herein can be used in diagnostic assays; to generate antibodies. Further, the antibodies and fragments thereof can also be used in diagnostic assays, to produce immunogenic compositions or the like.

Before describing the present invention in detail, it is to be understood that this invention is not limited to particularly exemplified molecules or process parameters as such may, of course, vary. It is also to be understood that the terminology used herein is for the purpose of describing particular embodiments of the invention only, and is not intended to be limiting. In addition, the practice of the present invention will employ, unless otherwise indicated, conventional methods of plant biology, virology, microbiology, molecular biology, recombinant DNA techniques and immunology all of which are within the ordinary skill of the art. Such techniques are explained fully in the literature. See, e.g., Evans, et al., Handbook of Plant Cell Culture (1983, Macmillan Publishing Co.); Binding, Regeneration of Plants, Plant Protoplasts (1985, CRC Press); Sambrook, et al., Molecular Cloning: A Laboratory Manual (2nd Edition, 1989); DNA Cloning: A Practical Approach, vol. I & II (D. Glover, ed.); Oligonucleotide Synthesis (N. Gait, ed., 1984); A Practical Guide to Molecular Cloning (1984); and Fundamental Virology, 2nd Edition, vol. I & II (B. N. Fields and D. M. Knipe, eds.).

All publications, patents and patent applications cited herein, whether supra or infra, are hereby incorporated by reference in their entirety.

It must be noted that, as used in this specification and the appended claims, the singular forms “a”, “an” and “the” include plural referents unless the content clearly dictates otherwise. Thus, for example, reference to “a polypeptide” includes a mixture of two or more polypeptides, and the like.

The following amino acid abbreviations are used throughout the text: TABLE-US-00001 Alanine: Ala (A) Arginine: Arg (R) Asparagine: Asn (N) Aspartic acid: Asp (D) Cysteine: Cys (C) Glutamine: Gln (Q) Glutamic acid: Glu (E) Glycine: Gly (G) Histidine: His (H) Isoleucine: Ile (I) Leucine: Leu (L) Lysine: Lys (K) Methionine: Met (M) Phenylalanine: Phe (F) Proline: Pro (P) Serine: Ser (S) Threonine: Thr (T) Tryptophan: Trp (W) Tyrosine: Tyr (Y) Valine: Val (V)

In describing the present invention, the following terms will be employed, and are intended to be defined as indicated below.

Definitions

The terms “nucleic acid molecule” and “polynucleotide” are used interchangeably and refer to a polymeric form of nucleotides of any length, either deoxyribonucleotides or ribonucleotides, or analogs thereof. This term refers only to the primary structure of the molecule and thus includes double- and single-stranded DNA and RNA. It also includes known types of modifications, for example, labels which are known in the art, methylation, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example proteins (including e.g., nucleases, toxins, antibodies, signal peptides, poly-L-lysine, etc.), those with intercalators (e.g., acridine, psoralen, etc.), those containing chelates (e.g., metals, radioactive metals, boron, oxidative metals, etc.), those containing alkylators, those with modified linkages (e.g., alpha anomeric nucleic acids, etc.), as well as unmodified forms of the polynucleotide. Polynucleotides may have any three-dimensional structure, and may perform any function, known or unknown. Nonlimiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term polynucleotide sequence is the alphabetical representation of a polynucleotide molecule. This alphabetical representation can be input into databases in a computer having a central processing unit and used for bioinformatics applications such as functional genomics and homology searching.

Techniques for determining nucleic acid and amino acid “sequence identity” are known in the art. Typically, such techniques include determining the nucleotide sequence of the mRNA for a gene and/or determining the amino acid sequence encoded thereby, and comparing these sequences to a second nucleotide or amino acid sequence. In general, “identity” refers to an exact nucleotide-to-nucleotide or amino acid-to-amino acid correspondence of two polynucleotides or polypeptide sequences, respectively. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMRL+DDBJ+PDB+GenBank CDS translations+Swiss protein+Spupdate+PIR.

Alternatively, the degree of sequence similarity between polynucleotides can be determined by hybridization of polynucleotides under conditions that form stable duplexes between homologous regions, followed by digestion with single-stranded-specific nuclease(s), and size determination of the digested fragments. Two DNA, or two polypeptide sequences are “substantially homologous” to each other when the sequences exhibit at least about 43%-60%, preferably 60-70%, more preferably 70%-85%, more preferably at least about 85%-90%, more preferably at least about 90%-95%, and most preferably at least about 95%-98% sequence identity over a defined length of the molecules, or any percentage between the above-specified ranges, as determined using the methods above. As used herein, substantially homologous also refers to sequences showing complete identity to the specified DNA or polypeptide sequence, DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

The degree of sequence identity between two nucleic acid molecules affects the efficiency and strength of hybridization events between such molecules. A partially identical nucleic acid sequence will at least partially inhibit a completely identical sequence from hybridizing to a target molecule. Inhibition of hybridization of the completely identical sequence can be assessed using hybridization assays that are well known in the art (e.g., Southern blot, Northern blot, solution hybridization, or the like, see Sambrook, et al., Molecular Cloning: A laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a target nucleic acid sequence, and then by selection of appropriate conditions the probe and the target sequence “selectively hybridize,” or bind, to each other to form a hybrid molecule. A nucleic acid molecule that is capable of hybridizing selectively to a target sequence under “moderately stringent” typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/target hybridization where the probe and target have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press).

With respect to stringency conditions for hybridization, it is well known in the art that numerous equivalent conditions can be employed to establish a particular stringency by varying, for example, the following factors: the length and nature of probe and target sequences, base composition of the various sequences, concentrations of salts and other hybridization solution components, the presence or absence of blocking agents in the hybridization solutions (e.g., formamide, dextran sulfate, and polyethylene glycol), hybridization reaction temperature and time parameters, as well as, varying wash conditions. The selection of a particular set of hybridization conditions is selected following standard methods in the art (see, for example, Sambrook, et al., Molecular Cloning: A Laboratory Manual, Second Edition, (1989) Cold Spring Harbor, N.Y.).

A “gene” as used in the context of the present invention is a sequence of nucleotides in a genetic nucleic acid (chromosome, plasmid, etc.) with which a genetic function is associated. A gene is a hereditary unit, for example of an organism, comprising a polynucleotide sequence that occupies a specific physical location (a “gene locus” or “genetic locus”) within the genome (nuclear or plastid genome) of an organism. A gene can encode an expressed product, such as a polypeptide or a polynucleotide (e.g., tRNA). Alternatively, a gene may define a genomic location for a particular event/function, such as the binding of proteins and/or nucleic acids, wherein the gene does not encode an expressed product. Typically, a gene includes coding sequences, such as, polypeptide encoding sequences, and non-coding sequences, such as, promoter sequences, polyadenylation sequences, transcriptional regulatory sequences (e.g., enhancer sequences). Many eucaryotic genes have “exons” (coding sequences) interrupted by “introns” (non-coding sequences). In certain cases, a gene may share sequences with another gene(s) (e.g., overlapping genes). In the context of the present invention, a gene also pertains to fragments or variants of known genes encoding enzymes wherein the fragment or variant encodes a polypeptide that retains the enzymatic activity.

A ‘coding sequence’ or a sequence which “encodes” a selected polypeptide, is a nucleic acid molecule which is transcribed (in the case of DNA) and translated (in the case of mRNA) into a polypeptide, for example, in vivo when placed under the control of appropriate regulatory sequences (or “control elements”). The boundaries of the coding sequence are typically determined by a start codon at the 5′ (amino) terminus and a translation stop codon at the 3′ (carboxy) terminus. A coding sequence can include, but is not limited to, cDNA from viral, procaryotic or eucaryotic mRNA, genomic DNA sequences from viral or procaryotic DNA, and even synthetic DNA sequences. A transcription termination sequence may be located 3′ to the coding sequence. Other “control elements” may also be associated with a coding sequence. A DNA sequence encoding a polypeptide can be optimized for expression in a selected cell by using the codons preferred by the selected cell to represent the DNA copy of the desired polypeptide coding sequence. “Encoded by” refers to a nucleic acid sequence which codes for a polypeptide sequence, wherein the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence. Also encompassed are polypeptide sequences which are immunologically identifiable with a polypeptide encoded by the sequence.

Typical “control elements”, include, but are not limited to, transcription promoters, transcription enhancer elements, transcription termination signals, polyadenylation sequences (located 3′ to the translation stop codon), sequences for optimization of initiation of translation (located 5′ to the coding sequence), translation enhancing sequences, and translation termination sequences. Transcription promoters can include inducible promoters (where expression of a polynucleotide sequence operably linked to the promoter is induced by an analyte, cofactor, regulatory protein, etc.), tissue-specific promoters (where expression of a polynucleotide sequence operably linked to the promoter is induced only in selected tissue), repressible promoters (where expression of a polynucleotide sequence operably linked to the promoter is induced by an analyte, cofactor, regulatory protein, etc.), and constitutive promoters.

A control element, such as a promoter, “directs the transcription” of a coding sequence in a cell when RNA polymerase will bind the promoter and transcribe the coding sequence into mRNA, which is then translated into the polypeptide encoded by the coding sequence.

“Expression enhancing sequences” typically refer to control elements that improve transcription or translation of a polynucleotide relative to the expression level in the absence of such control elements (for example, promoters, promoter enhancers, enhancer elements, and translational enhancers (e.g., Shine and Delagarno sequences).

“Operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences. The control elements need not be contiguous with the coding sequence, so long as they function to direct the expression thereof. Thus, for example, intervening untranslated yet transcribed sequences can be present between a promoter and the coding sequence and the promoter can still be considered “operably linked” to the coding sequence.

A “heterologous sequence” as used herein typically refers to a nucleic acid sequence that is not normally found in the cell or organism of interest. For example, a DNA sequence encoding a polypeptide can be obtained from a plant cell and introduced into a bacterial cell. In this case the plant DNA sequence is “heterologous” to the native DNA of the bacterial cell.

“Recombinant” as used herein to describe a nucleic acid molecule means a polynucleotide of genomic, cDNA, semisynthetic, or synthetic origin which, by virtue of its origin or manipulation: (1) is not associated with all or a portion of the polynucleotide with which it is associated in nature; and/or (2) is linked to a polynucleotide other than that to which it is linked in nature. The term “recombinant” as used with respect to a protein or polypeptide means a polypeptide produced by expression of a recombinant polynucleotide.

By “vector” is meant any genetic element, such as a plasmid, phage, transposon, cosmid, chromosome, virus etc., which is capable of transferring gene sequences to target cells. Generally, a vector is capable of replication when associated with the proper control elements. Thus, the term includes cloning and expression vehicles, as well as viral vectors and integrating vectors.

As used herein, the term “expression cassette” refers to a molecule comprising at least one coding sequence, optionally also operably linked to a control sequence, which includes all nucleotide sequences required for the transcription of cloned copies of the coding sequence and the translation of the mRNAs in an appropriate host cell. Such expression cassettes can be used to express eukaryotic genes in a variety of hosts such as bacteria, blue-green algae, plant cells, yeast cells, insect cells and animal cells, either in vivo or in vitro. Under the invention, expression cassettes can include, but are not limited to, cloning vectors, specifically designed plasmids, viruses or virus particles. The cassettes may further include an origin of replication for autonomous replication in host cells, selectable markers, various restriction sites, a potential for high copy number and strong promoters.

A cell has been “transformed” by an exogenous polynucleotide when the polynucleotide has been introduced inside the cell. The exogenous polynucleotide may or may not be integrated (covalently linked) into chromosomal DNA making up the nuclear or plastid genome of the cell. In prokaryotes and yeasts, for example, the exogenous DNA may be maintained on an episomal element, such as a plasmid. With respect to eucaryotic cells, a stably transformed cell is one in which the exogenous DNA has become integrated into the chromosome so that it is inherited by daughter cells through chromosome replication. This stability is demonstrated by the ability of the eucaryotic cell to establish cell lines or clones comprised of a population of daughter cells containing the exogenous DNA.

“Recombinant host cells,” “host cells,” “cells,” “cell lines,” “cell cultures,” and other such terms denoting procaryotic microorganisms or eucaryotic cell lines cultured as unicellular entities, are used interchangeably, and refer to cells which can be, or have been, used as recipients for recombinant vectors or other transfer DNA, and include the progeny of the original cell which has been transfected. It is understood that the progeny of a single parental cell may not necessarily be completely identical in morphology or in genomic or total DNA complement to the original parent, due to accidental or deliberate mutation. Progeny of the parental cell which are sufficiently similar to the parent to be characterized by the relevant property, such as the presence of a nucleotide sequence encoding a desired peptide, are included in the progeny intended by this definition, and are covered by the above terms.

The term “Bgl gene” refers to known polynucleotides that encode a beta-glucoside glucohydrolase enzyme, or optionally, functional polypeptide analogs and/or fragments. Examples of Bgl genes that may be implemented in accordance with the teachings herein include, but are not limited to, Accession nos. U09580, FJ882071, Z7ACA8, XM_{—}002422368.1, Q6UJY0, Q5QMT0, Q3ECW8, and AE005674.1, and others. In specific embodiments, the Bgl gene is a Bgl1 gene, which encodes a beta-glucosidase 1.

The term “phenotype” as used herein refers to any microscopic or macroscopic change in structure or morphology of a plant, such as a transgenic plant, as well as biochemical differences, which are characteristic of a plant with increased phytohormone content, compared to a progenitor, wild-type plant cultivated under the same conditions. Generally, such morphological differences include increase of apical dominance, increased hypocotyl length, increased biomass, increased height, and/or increased number and/or size of trichomes.

A “polypeptide” is used in its broadest sense to refer to a compound of two or more subunit amino acids, amino acid analogs, or other peptidomimetics. The subunits may be linked by peptide bonds or by other bonds, for example ester, ether, etc. As used herein, the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is typically called a polypeptide or a protein. Full-length proteins, analogs, mutants and fragments thereof are encompassed by the definition. The terms also include postexpression modifications of the polypeptide, for example, glycosylation, acetylation, phosphorylation and the like. Furthermore, as ionizable amino and carboxyl groups are present in the molecule, a particular polypeptide may be obtained as an acidic or basic salt, or in neutral form. A polypeptide may be obtained directly from the source organism, or may be recombinantly or synthetically produced (see further below).

A “Bgl polypeptide” is a polypeptide as defined above, which is expressed from a Bgl gene and that retains beta-glucosidase enzymatic activity. The term encompasses mutants and fragments of the native sequence so long as the protein functions for its intended purpose.

The term “Bgl analog” refers to derivatives of a Bgl polypeptide, or fragments of such derivatives, that retain desired function, e.g., as measured in assays as described further below. In general, the term “analog” refers to compounds having a native polypeptide sequence and structure with one or more amino acid additions, substitutions (generally conservative in nature) and/or deletions, relative to the native molecule, so long as the modifications do not destroy desired activity. Preferably, the analog has at least the same activity as the native molecule. Methods for making polypeptide analogs are known in the art and are described further below.

Particularly preferred analogs include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, trosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. It is to be understood that the terms include the various sequence polymorphisms that exist, wherein amino acid substitutions in the protein sequence do not affect the essential functions of the protein.

By “purified” and “isolated” is meant, when referring to a polypeptide or polynucleotide, that the molecule is separate and discrete from the whole organism with which the molecule is found in nature; or devoid, in whole or part, of sequences normally associated with it in nature; or a sequence, as it exists in nature, but having heterologous sequences (as defined below) in association therewith. It is to be understood that the term “isolated” with reference to a polynucleotide intends that the polynucleotide is separate and discrete from the chromosome from which the polynucleotide may derive. The term “purified” as used herein preferably means at least 75% by weight, more preferably at least 85% by weight, more preferably still at least 95% by weight, and most preferably at least 98% by weight, of biological macromolecules of the same type are present. An “isolated polynucleotide which encodes a particular polypeptide” refers to a nucleic acid molecule which is substantially free of other nucleic acid molecules that do not encode the subject polypeptide; however, the molecule may include some additional bases or moieties which do not deleteriously affect the basic characteristics of the composition.

By “fragment” is intended a polypeptide or polynucleotide consisting of only a part of the intact sequence and structure of the reference polypeptide or polynucleotide, respectively. The fragment can include a 3′ or C-terminal deletion or a 5′ or N-terminal deletion, or even an internal deletion, of the native molecule. A polynucleotide fragment of a Bgl sequence will generally include at least about 15 contiguous bases of the molecule in question, more preferably 18-25 contiguous bases, even more preferably 30-50 or more contiguous bases of the Bgl molecule, or any integer between 15 bases and the full-length sequence of the molecule. Fragments which provide at least one Bgl phenotype as defined above are useful in the production of transgenic plants. Fragments are also useful as oligonucleotide probes, to find additional Bgl sequences, e.g., in different plant species.

Similarly, a polypeptide fragment of a Bgl polypeptide will generally include at least about 5-10 contiguous amino acid residues of the full-length molecule, preferably at least about 15-25 contiguous amino acid residues of the full-length molecule, and most preferably at least about 20-50 or more contiguous amino acid residues of the full-length Bgl polypeptide molecule, or any integer between 10 amino acids and the full-length sequence of the molecule. Such fragments are useful for the production of antibodies and the like.

By “transgenic plant” is meant a plant into which one or more exogenous polynucleotides have been introduced. Examples of means by which this can be accomplished are described below, and include Agrobacterium-mediated transformation, biolistic methods, electroporation, and the like. In the context of the present invention, the transgenic plant contains a Bgl gene polynucleotide which is over-expressed (i.e., contains increased expression of the Bgl gene relative to wild-type plant) and which confers at least one phenotypic trait to the plant, as defined above. The transgenic plant therefore exhibits altered structure, morphology or biochemistry as compared with a progenitor plant which does not contain the transgene, when the transgenic plant and the progenitor plant are cultivated under similar or equivalent growth conditions. A transgenic plant may also over- or underexpress glucosinolates. Such a plant containing the exogenous polynucleotide is referred to here as an R₁ generation transgenic plant. Transgenic plants may also arise from sexual cross or by selfing of transgenic plants into which exogenous polynucleotides have been introduced. Such a plant containing the exogenous nucleic acid is also referred to here as an R₁ generation transgenic plant. Transgenic plants which arise from a sexual cross with another parent line or by selfing are “descendants or the progeny” of a R₁ plant and are generally called Fₙ plants or Sₙ plants, respectively, n meaning the number of generations.

A “vacuole targeting sequence” is a polynucleotide sequence encoding a vacuole targeting peptide that can be linked to a Bgl gene such that the Bgl polypeptide linked to the vacuole targeting peptide is directed or sorted to a plant vacuole. Examples of vacuole targeting sequences include but are not limited to a C-terminal propeptide (CTPP) of Concanavalin A, Chitinase A and/or N-terminal propeptide (NTPP) of sporamin; those described U.S. Patent App 20090193541; and others known in the art. In other examples, the vacuole targeting sequence may be a leader peptide of a strictosidine synthase gene, e.g. that of the Catharanthus roseus strictosidine synthase (McKnight et al., Nucleic Acids Research (1990), 18, 4939; incorporated herein by reference) or of Rauwolfia serpentine strictisodine synthase (Kutchan et al. (1988) FEBS Lett 237 40-44; incorporated herein by reference). For a review of vacuole targeting sequences see Neuhaus (1996) Plant Physiol Biochem 34(2) 217-221.

Other sequences which may be linked to the gene of interest which encodes a polypeptide are those which can target to a specific organelle, e.g., to the mitochondria, nucleus, or plastid, within the plant cell. Targeting can be achieved by providing the polypeptide with an appropriate targeting peptide sequence, such as a secretory signal peptide (for secretion or cell wall or membrane targeting, a plastid transit peptide, a chloroplast transit peptide, e.g., the chlorophyll a/b binding protein, a mitochondrial target peptide, or a nuclear targeting peptide, and the like. For example, the small subunit of ribulose bisphosphate carboxylase transit peptide, the EPSPS transit peptide or the dihydrodipicolinic acid synthase transit peptide may be used. For examples of plastid organelle targeting sequences (see WO 00/12732).

General Overview

The inventors herein have discovered that overexpression of beta-glucosidase in plants results in an increase of phytohormone in plants. This in turn leads to desirous agronomic traits, including increased biomass, increased height early flowering, trichome production and parasite resistance. Plants which overexpress or underexpress this enzyme, therefore, have altered phenotypes, as described above. Thus, plant growth, nutritional values and plant pathogens can be affected by modulating levels of expression of this enzyme.

The present invention particularly provides for altered structure or morphology such as increased size of leaves or fruit, increased branching, increased seed production, increased trichome production, and increased parasite resistance relative wild-type plants. Heterologous Bgl genes can be expressed to engineer a plant with desirable properties. The engineering is accomplished by transforming plants with nucleic acid constructs described herein which may also comprise promoters and secretion signal peptides. The transformed plants or their progenies are screened for plants that express the desired polypeptide.

Engineered plants exhibiting the desired altered structure or morphology can be used in plant breeding or directly in agricultural production or industrial applications. Plants having the altered phenotypes can be crossed with other altered plants engineered with alterations in other growth modulation enzymes, proteins or polypeptides to produce lines with even further enhanced altered structural morphology characteristics compared to the parents or progenitor plants.

Isolation of Nucleic Acid Sequences from Plants

The isolation of Bgl genes may be accomplished by a number of techniques. For instance, oligonucleotide probes based on the sequences disclosed herein can be used to identify the desired gene in a cDNA or genomic DNA library from a desired species. To construct genomic libraries, large segments of genomic DNA are generated by random fragmentation, e.g. using restriction endonucleases, and are ligated with vector DNA to form concatemers that can be packaged into the appropriate vector. To prepare a library of tissue-specific cDNAs, mRNA is isolated from tissues and a cDNA library which contains the gene transcripts is prepared from the mRNA.

The cDNA or genomic library can then be screened using a probe based upon the sequence of a cloned gene such as the polynucleotides disclosed here. Probes may be used to hybridize with genomic DNA or cDNA sequences to isolate homologous genes in the same or different plant species. Alternatively, the nucleic acids of interest can be amplified from nucleic acid samples using amplification techniques. For instance, polymerase chain reaction (PCR) technology to amplify the sequences of the genes directly from mRNA, from cDNA, from genomic libraries or cDNA libraries. PCR® and other in vitro amplification methods may also be useful, for example, to clone nucleic acid sequences that code for proteins to be expressed, to make nucleic acids to use as probes for detecting the presence of the desired mRNA in samples, for nucleic acid sequencing, or for other purposes.

Appropriate primers and probes for identifying Bgl-specific genes from tissues are generated from comparisons of the sequences provided herein. For a general overview of PCR see Innis et al. eds, PCR Protocols: A Guide to Methods and Applications, Academic Press, San Diego (1990). Suitable amplifications conditions may be readily determined by one of skill in the art in view of the teachings herein, for example, including reaction components and amplification conditions as follows: 10 mM Tris-HCl, pH 8.3, 50 mM potassium chloride, 1.5 mM magnesium chloride, 0.001% gelatin, 200 μM dATP, 200 μM dCTP, 200 μM dGTP, 200 μM dTTP, 0.4 μM primers, and 100 units per mL Taq polymerase; 96° C. for 3 min., 30 cycles of 96° C. for 45 seconds, 50° C. for 60 seconds, 72° C. for 60 seconds, followed by 72° C. for 5 min.

Polynucleotides may also be synthesized by well-known techniques as described in the technical literature. See, e.g., Carruthers, et al. (1982) Cold Spring Harbor Symp. Quant. Biol. 47:411-418, and Adams, et al. (1983) J. Am. Chem. Soc. 105:661. Double stranded DNA fragments may then be obtained either by synthesizing the complementary strand and annealing the strands together under appropriate conditions, or by adding the complementary strand using DNA polymerase with an appropriate primer sequence.

The polynucleotides of the present invention may also be used to isolate or create other mutant cell gene alleles. Mutagenesis consists primarily of site-directed mutagenesis followed by phenotypic testing of the altered gene product. Some of the more commonly employed site-directed mutagenesis protocols take advantage of vectors that can provide single stranded as well as double stranded DNA, as needed. Generally, the mutagenesis protocol with such vectors is as follows. A mutagenic primer, i.e., a primer complementary to the sequence to be changed, but consisting of one or a small number of altered, added, or deleted bases, is synthesized. The primer is extended in vitro by a DNA polymerase and, after some additional manipulations, the now double-stranded DNA is transfected into bacterial cells. Next, by a variety of methods, the desired mutated DNA is identified, and the desired protein is purified from clones containing the mutated sequence. For longer sequences, additional cloning steps are often required because long inserts (longer than 2 kilobases) are unstable in those vectors. Protocols are known to one skilled in the art and kits for site-directed mutagenesis are widely available from biotechnology supply companies, for example from Amersham Life Science, Inc. (Arlington Heights, Ill.) and Stratagene Cloning Systems (La Jolla, Calif.).

Control Elements

Sequences controlling eukaryotic gene expression have been extensively studied. For instance, promoter sequence elements include the TATA box consensus sequence (TATAAT), which is usually 20 to 30 base pairs upstream of the transcription start site. In most instances the TATA box is required for accurate transcription initiation. In plants, further upstream from the TATA box, at positions −80 to −100, there is typically a promoter element with a series of adenines surrounding the trinucleotide G (or T) N G. (See, J. Messing et al., in Genetic Engineering in Plants, pp. 221-227 (Kosage, Meredith and Hollaender, eds. (1983)). Methods for identifying and characterizing promoter regions in plant genomic DNA are described, for example, in Jordano et al. (1989) Plant Cell 1:855-866; Bustos et al (1989) Plant Cell 1:839-854; Green et al. (1988) EMBO J. 7:4035-4044; Meier et al. (1991) Plant Cell 3:309-316; and Zhang et al (1996) Plant Physiology 110:1069-1079).

Additionally, the promoter region may include nucleotide substitutions, insertions or deletions that do not substantially affect the binding of relevant DNA binding proteins and hence the promoter function. It may, at times, be desirable to decrease the binding of relevant DNA binding proteins to “silence” or “down-regulate” a promoter, or conversely to increase the binding of relevant DNA binding proteins to “enhance” or “up-regulate” a promoter. In such instances, the nucleotide sequence of the promoter region may be modified by, e.g., inserting additional nucleotides, changing the identity of relevant nucleotides, including use of chemically-modified bases, or by deleting one or more nucleotides.

Promoter function can be assayed by methods known in the art, preferably by measuring activity of a reporter gene operatively linked to the sequence being tested for promoter function. Examples of reporter genes include those encoding luciferase, green fluorescent protein, GUS, neo, cat and bar.

Polynucleotides comprising untranslated (OR) sequences and intron/exon junctions may also be identified. UTR sequences include introns and 5′ or 3′ untranslated regions (5′ UTRs or 3′ UTRs). These portions of the gene, especially UTRs, can have regulatory functions related to, for example, translation rate and mRNA stability. Thus, these portions of the gene can be isolated for use as elements of gene constructs for expression of polynucleotides encoding desired polypeptides.

Introns of genomic DNA segments may also have regulatory functions. Sometimes promoter elements, especially transcription enhancer or suppressor elements, are found within introns. Also, elements related to stability of heteronuclear RNA and efficiency of transport to the cytoplasm for translation can be found in intron elements. Thus, these segments can also find use as elements of expression vectors intended for use to transform plants.

The introns, UTR sequences and intron/exon junctions can vary from the native sequence. Such changes from those sequences preferably will not affect the regulatory activity of the UTRs or intron or intron/exon junction sequences on expression, transcription, or translation. However, in some instances, down-regulation of such activity may be desired to modulate traits or phenotypic or in vitro activity.

In specific embodiments, regulatory regions can be isolated from a Bgl gene and used in recombinant constructs for modulating the expression of the gene or a heterologous gene in vitro and/or in vivo. This region may be used in its entirety or fragments of the region may be isolated which provide the ability to direct expression of a coding sequence linked thereto.

Thus, promoters can be identified by analyzing the 5′ sequences of a genomic clone including the Bgl gene and sequences characteristic of promoter sequences can be used to identify the promoter.

Use of Nucleic Acids of the Invention to Enhance Gene Expression

It will be apparent that the polynucleotides described herein can be used in a variety of combinations. For example, the polynucleotides can be used to produce different phenotypes in the same organism, for instance by using tissue-specific promoters to overexpress a Bgl polynucleotide in certain tissues (e.g., leaf tissue) while at the same time using tissue-specific promoters to inhibit expression of in other tissues. In addition, fusion proteins of the polynucleotides described herein with other known polynucleotides (e.g., polynucleotides encoding products involved in the brassinosteroid pathway) can be constructed and employed to obtain desired phenotypes.

Any of the polynucleotides described herein can also be used in standard diagnostic assays, for example, in assays for mRNA levels (see, Sambrook et al, supra); as hybridization probes, e.g., in combination with appropriate means, such as a label, for detecting hybridization (see, Sambrook et al., supra); as primers, e.g., for PCR (see, Sambrook et al., supra); attached to solid phase supports and the like.

Preparation of Recombinant Vectors

To use isolated sequences in the above techniques, recombinant DNA vectors suitable for transformation of plant cells are prepared. Techniques for transforming a wide variety of higher plant species are well known and described further below as well as in the technical and scientific literature. See, for example, Weising et al (1988) Ann. Rev. Genet. 22:421-477. A DNA sequence coding for the desired polypeptide, for example a cDNA sequence encoding the full-length Bgl protein, will preferably be combined with transcriptional and translational initiation regulatory sequences which will direct the transcription of the sequence from the gene in the intended tissues of the transgenic plant.

Such regulatory elements include but are not limited to the promoters derived from the genome of plant cells (e.g., heat shock promoters such as soybean hsp17.5-E or hsp17.3-B (Gurley et al. (1986) Mol. Cell. Biol. 6:559-565); the promoter for the small subunit of RUBISCO (Coruzzi et al. (1984) EMBO J. 3:1671-1680; Broglie et al (1984) Science 224:838-843); the promoter for the chlorophyll a/b binding protein) or from plant viruses viral promoters such as the 35S RNA and 19S RNA promoters of CaMV (Brisson et al. (1984) Nature 310:511-514), or the coat protein promoter of TMV (Takamatsu et al. (1987) EMBO J. 6:307-311), cytomegalovirus hCMV immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, heat shock promoters (e.g., as described above) and the promoters of the yeast alpha-mating factors.

In construction of recombinant expression cassettes of the invention, a plant promoter fragment may be employed which will direct expression of the gene in all tissues of a regenerated plant. Such promoters are referred to herein as “constitutive” promoters and are active under most environmental conditions and states of development or cell differentiation. Examples of constitutive promoters include the cauliflower mosaic virus (CaMV) 35S transcription initiation region, the T-DNA mannopine synthetase promoter (e.g., the 1′- or 2′-promoter derived from T-DNA of Agrobacterium tumafaciens), and other transcription initiation regions from various plant genes known to those of skill.

Alternatively, the plant promoter may direct expression of the polynucleotide of the invention in a specific tissue (tissue-specific promoters) or may be otherwise under more precise environmental control (inducible promoters). Examples of tissue-specific promoters under developmental control include promoters that initiate transcription only in certain tissues, such as fruit, seeds, or flowers such as tissue- or developmental-specific promoter, such as, but not limited to the CHS promoter, the PATATIN promoter, etc. The tissue specific E8 promoter from tomato is particularly useful for directing gene expression so that a desired gene product is located in fruits.

Other suitable promoters include those from genes encoding embryonic storage proteins. Examples of environmental conditions that may affect transcription by inducible promoters include anaerobic conditions, elevated temperature, or the presence of light. If proper polypeptide expression is desired, a polyadenylation region at the 3′-end of the coding region should be included. The polyadenylation region can be derived from the natural gene, from a variety of other plant genes, or from T-DNA. In addition, the promoter itself can be derived from the Bgl gene, as described above.

The vector comprising the sequences (e.g., promoters or coding regions) from Bgl will typically comprise a marker gene which confers a selectable phenotype on plant cells. For example, the marker may encode biocide resistance, particularly antibiotic resistance, such as resistance to kanamycin, G418, bleomycin, hygromycin, or herbicide resistance, such as resistance to chlorosluforon or Basta.

Production of Transgenic Plants

DNA constructs may be introduced into the genome of the desired plant host by a variety of conventional techniques. For reviews of such techniques see, for example, Weissbach & Weissbach Methods for Plant Molecular Biology (1988, Academic Press, N.Y.) Section VIII, pp. 421-463; and Grierson & Corey, Plant Molecular Biology (1988, 2d Ed.), Blackie, London, Ch. 7-9. For example, the DNA construct may be introduced directly into the genomic DNA of the plant cell using techniques such as electroporation and microinjection of plant cell protoplasts, or the DNA constructs can be introduced directly to plant tissue using biolistic methods, such as DNA particle bombardment (see, e.g., Klein et al (1987) Nature 327:70-73). Alternatively, the DNA constructs may be combined with suitable T-DNA flanking regions and introduced into a conventional Agrobacterium tumefaciens host vector. Agrobacterium tumefaciens-mediated transformation techniques, including disarming and use of binary vectors, are well described in the scientific literature. See, for example Horsch et al (1984) Science 233:496-498, and Fraley et al (1983) Proc. Nat'l. Acad. Sci. USA 80:4803. The virulence functions of the Agrobacterium tumefaciens host will direct the insertion of the construct and adjacent marker into the plant cell DNA when the cell is infected by the bacteria using binary T DNA vector (Bevan (1984) Nuc. Acid Res. 12:8711-8721) or the co-cultivation procedure (Horsch et al (1985) Science 227:1229-1231). Generally, the Agrobacterium transformation system is used to engineer dicotyledonous plants (Bevan et al (1982) Ann. Rev. Genet 16:357-384; Rogers et al (1986) Methods Enymol. 118:627-641). The Agrobacterium transformation system may also be used to transform, as well as transfer, DNA to monocotyledonous plants and plant cells. (see Hernalsteen et al (1984) EMBO J 3:3039-3041; Hooykass-Van Slogteren et al (1984) Nature 311:763-764; Grimsley et al (1987) Nature 325:1677-179; Boulton et al (1989) Plant Mol. Biol. 12:31-40; and Gould et al (1991) Plant Physiol. 95:426-434).

Alternative gene transfer and transformation methods include, but are not limited to, protoplast transformation through calcium-, polyethylene glycol (PEG)- or electroporation-mediated uptake of naked DNA (see Paszkowski et al. (1984) EMBO J 3:2717-2722, Potrykus et al. (1985) Molec. Gen. Genet. 199:169-177; Fromm et al. (1985) Proc. Nat. Acad. Sci. USA 82:5824-5828; and Shimamoto (1989) Nature 338:274-276) and electroporation of plant tissues (D'Halluin et al. (1992) Plant Cell 4:1495-1505). Additional methods for plant cell transformation include microinjection, silicon carbide mediated DNA uptake (Kaeppler et al. (1990) Plant Cell Reporter 9:415-418), and microprojectile bombardment (see Klein et al. (1988) Proc. Nat. Acad. Sci. USA 85:4305-4309; and Gordon-Kamm et al. (1990) Plant Cell 2:603-618).

Transformed plant cells which are produced by any of the above transformation techniques can be cultured to regenerate a whole plant which possesses the transformed genotype and thus the desired phenotype. Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is described in Evans, et al., “Protoplasts Isolation and Culture” in Handbook of Plant Cell Culture, pp. 124-176, Macmillian Publishing Company, New York, 1983; and Binding, Regeneration of Plants, Plant Protoplasts, pp. 21-73, CRC Press, Boca Raton, 1985. Regeneration can also be obtained from plant callus, explants, organs, pollens, embryos or parts thereof. Such regeneration techniques are described generally in Klee et al (1987) Ann. Rev. of Plant Phys. 38:467-486.

The nucleic acids of the invention can be used to confer desired traits on essentially any plant. A wide variety of plants and plant cell systems may be engineered for the desired physiological and agronomic characteristics described herein using the nucleic acid constructs of the present invention and the various transformation methods mentioned above. In preferred embodiments, target plants and plant cells for engineering include, but are not limited to, those monocotyledonous and dicotyledonous plants, such as crops including grain crops (e.g., wheat, maize, rice, millet, barley), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed) and plants used for experimental purposes (e.g., Arabidopsis). Thus, the invention has use over a broad range of plants, including, but not limited to, species from the genera Asparagus, Avena, Brassica, Citrus, Citrullus, Capsicum, Cucurbita, Daucus, Glycine, Hordeum, Lactuca, Lycopersicon, Malus, Manilot, Nicotiana, Oryza, Persea, Pisum, Pyrus, Prunus, Raphanus, Secale, Solanum, Sorghum, Triticum, Vitis, Vigna, and Zea. Also, crops such as Cannabis sativa, Papaver somniferum or Erythorxylum coca may be transformed to increase biomass or trichome production.

One of skill in the art will recognize that after the expression cassette is stably incorporated in transgenic plants and confirmed to be operable, it can be introduced into other plants by sexual crossing. Any of a number of standard breeding techniques can be used, depending upon the species to be crossed.

A transformed plant cell, callus, tissue or plant may be identified and isolated by selecting or screening the engineered plant material for traits encoded by the marker genes present on the transforming DNA. For instance, selection may be performed by growing the engineered plant material on media containing an inhibitory amount of the antibiotic or herbicide to which the transforming gene construct confers resistance. Further, transformed plants and plant cells may also be identified by screening for the activities of any visible maker genes (e.g., the β-glucuronidase, luciferase, B or Cl genes) that may be present on the recombinant nucleic acid constructs of the present invention. Such selection and screening methodologies are well known to those skilled in the art.

Physical and biochemical methods also may be used to identify plant or plant cell transformants containing the gene constructs of the present invention. These methods include but are not limited to: 1) Southern analysis or PCR amplification for detecting and determining the structure of the recombinant DNA insert; 2) Northern blot, S1 RNase protection, primer-extension or reverse transcriptase-PCR amplification for detecting and examining RNA transcripts of the gene constructs; 3) enzymatic assays for detecting enzyme or ribozyme activity, where such gene products are encoded by the gene construct; 4) protein gel electrophoresis, Western blot techniques, immunoprecipitation, or enzyme-linked immunoassays, where the gene construct products are proteins. Additional techniques, such as in situ hybridization, enzyme staining, and immunostaining, also may be used to detect the presence or expression of the recombinant construct in specific plant organs and tissues. The methods for doing all these assays are well known to those skilled in the art.

Effects of gene manipulation using the methods of this invention can be observed by, for example, northern blots of the RNA (e.g., RNA) isolated from the tissues of interest.

The transgene may be selectively expressed in some tissues of the plant or at some developmental stages, or the transgene may be expressed in substantially all plant tissues, substantially along its entire life cycle. However, any combinatorial expression mode is also applicable.

The present invention also encompasses seeds of the transgenic plants described above wherein the seed has the transgene or gene construct. The present invention further encompasses the progeny, clones, cell lines or cells of the transgenic plants described above wherein said progeny, clone, cell line or cell has the transgene or gene construct.

Polypeptides

The present invention also includes Bgl polypeptides, including such polypeptides as a fusion, or chimeric protein product (comprising the protein, fragment, analogue, mutant or derivative joined via a peptide bond to a heterologous protein sequence (of a different protein)). Such a chimeric product can be made by ligating the appropriate nucleic acid sequences encoding the desired amino acid sequences to each other by methods known in the art, in the proper coding frame, and expressing the chimeric product by methods commonly known in the art.

As noted above, the phenotype due to over or underexpression of a Bgl gene includes any macroscopic, microscopic or biochemical changes which are characteristic of release of increased β-glucosidase activity and/or increased phytohormone release. Thus, the phenotype (e.g., activities) can include any activity that is exhibited by the native Bgl polypeptide including, for example, in vitro, in vivo, biological, enzymatic, immunological, substrate binding activities, etc. Non-limiting examples of such activities include:

(a) activities displayed by other glucosidase enzymes;

(b) increase cellular phytohormone levels;

(c) regulation of glucosinolates, cytokines and auxins; and

(d) induce resistance to plant pathogens (see, e.g., U.S. Pat. No. 5,952,545).

A Bgl analog, whether a derivative, fragment or fusion of native Bgl polypeptides, is capable of at least one Bgl activity. Preferably, the analogs exhibit at least 60% of the activity of the native protein, more preferably at least 70% and even more preferably at least 80%, 85%, 90% or 95% of at least one activity of the native protein.

Further, such analogs exhibit some sequence identity to the native Bgl polypeptide sequence. Preferably, the variants will exhibit at least 35%, more preferably at least 59%, even more preferably 75% or 80% sequence identity, even more preferably 85% sequence identity, even more preferably, at least 90% sequence identity; more preferably at least 95%, 96%, 97%, 98% or 99% sequence identity.

Bgl analogs can include derivatives with increased or decreased activities as compared to the native Bgl polypeptides. Such derivatives can include changes within the domains, motifs and/or consensus regions of the native Bgl polypeptide.

One class of analogs is those polypeptide sequences that differ from the native Bgl polypeptide by changes, insertions, deletions, or substitution; at positions flanking the domain and/or conserved residues. For example, an analog can comprise (1) the domains of a Bgl polypeptide and/or (2) at conserved or nonconserved residues. For example, an analog can comprise residues conserved between the Bgl polypeptide and other beta-glucosidase proteins with other regions of the molecule changed.

Another class of analogs includes those that comprise a Bgl polypeptide sequence that differs from the native sequence in the domain of interest or conserved residues by a conservative substitution.

Fusion polypeptides comprising Bgl polypeptides (e.g., native, analogs, or fragments thereof) can also be constructed. Non-limiting examples of other polypeptides that can be used in fusion proteins include chimeras of Bgl polypeptides and fragments thereof.

In addition, Bgl polypeptides, derivatives (including fragments and chimeric proteins), mutants and analogues can be chemically synthesized. See, e.g., Clark-Lewis et al. (1991) Biochem. 30:3128-3135 and Merrifield (1963) J. Amer. Chem. Soc. 85:2149-2156. For example, Bgl, derivatives, mutants and analogues can be synthesized by solid phase techniques, cleaved from the resin, and purified by preparative high performance liquid chromatography (e.g., see Creighton, 1983, Proteins, Structures and Molecular Principles, W.H. Freeman and Co., N.Y., pp. 50-60). Bgl, derivatives and analogues that are proteins can also be synthesized by use of a peptide synthesizer. The composition of the synthetic peptides may be confirmed by amino acid analysis or sequencing (e.g., the Edman degradation procedure; see Creighton, 1983, Proteins, Structures and Molecular Principles, W.H. Freeman and Co., N.Y., pp. 34-49).

Further, the polynucleotides and polypeptides described herein can be used to generate antibodies that specifically recognize and bind to the protein products of the CYP83A1 polynucleotides. (See, Harlow and Lane, eds. (1988) “Antibodies: A Laboratory Manual”). The polypeptides and antibodies thereto can also be used in standard diagnostic assays, for example, radioimmunoassays, ELISA (enzyme linked immunoradiometric assays), “sandwich” immunoassays, immunoradiometric assays, in situ immunoassay, western blot analysis, immunoprecipitation assays, immunofluorescent assays and PAGE-SDS.

### EXAMPLES

### Example 1

### Chloroplast Transformation Vector

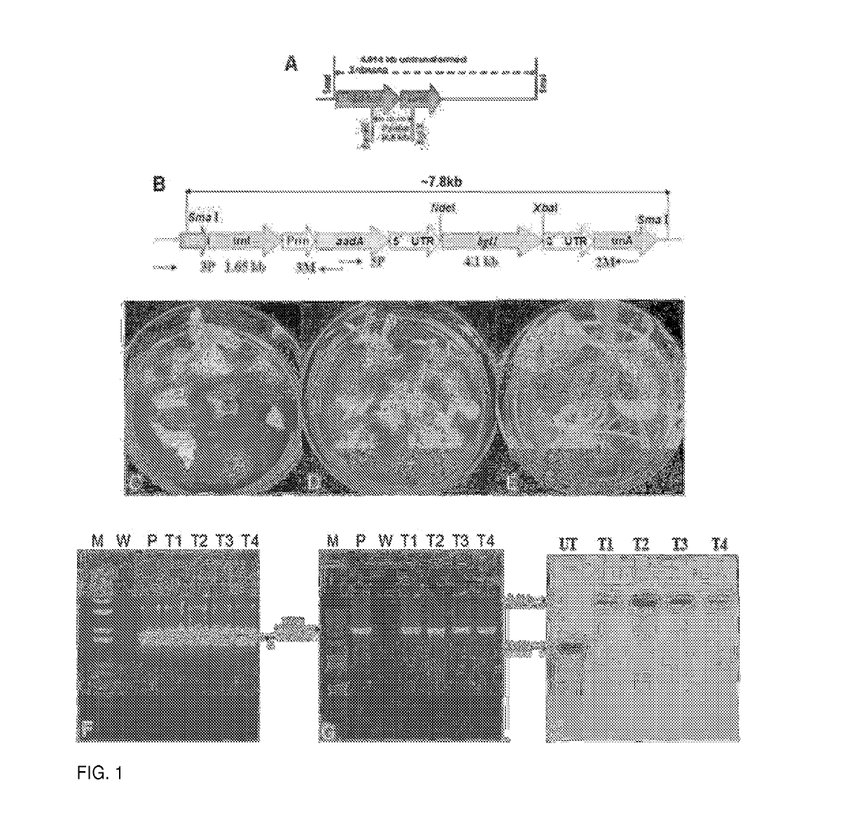

The coding sequence of the β-glucosidase gene (bgl1) was amplified from Trichoderma reesei genomic DNA using a PCR-based method (An et al., 2007). To create tobacco plants expressing β-glucosidase, leaf explants from tobacco were transformed with the chloroplast transformation vector (pLD) containing the bgl1 gene (FIG. 1B). Site-specific integration of bgl1 into the trnI/trnA spacer region of the chloroplast genome was achieved through the pLD vector containing the homologous recombination sequences as described previously (Verma and Daniell, 2007; Daniell et al., 2009). This site of integration has several unique advantages (Daniell et al., 2004). The psbA promoter/5′ untranslated region inserted upstream of the bgl-1 gene is anticipated to increase transcription and translation in the light, and the 3′ untranslated region is believed to increase transcript stability. The constitutive 16S rRNA promoter regulates expression of the aminoglycoside 3′ adenylyltransferase (aadA) gene to confer spectinomycin resistance.

### Example 2

### Integration of the Expression Cassette and Homoplasmy

After bombardment of the chloroplast integration and expression vector pLD-utr-bgl1, several spectinomycin-resistant shoots appeared from the bombarded tobacco leaves in the first round of selection (FIG. 1C). Homoplasmic shoots were obtained after the second round of selection (FIG. 1D). The third round of selection on half-strength Murashige and Skoog (MS) medium (FIG. 1E) established transplastomic lines. To confirm the integration of transgene cassettes into the chloroplast genome, the putative transformed plantlets were screened by PCR. Two pairs of primers were used for screening. The 3P and 3M primers were used to check integration of the selectable marker gene (aadA) into the chloroplast genome. The 5P and 2M primers were used to confirm integration of the transgene expression cassette. DNA samples from the BGL-1 shoots showed PCR-positive results with both primers (FIG. 1, F and G). The 3P-3M PCR product size for BGL-1 plants was 1.65 kb, and the 5P-2M PCR product size was 4.1 kb.

Southern-blot analysis was performed to investigate whether BGL-1 transplastomic lines achieved homoplasmy. The probe used was made by digesting the flanking sequences trnI and trnA with BamHI and BglII (FIG. 1A). Flanking sequence probe hybridized with a single 4.0-kb fragment in untransformed (UT) chloroplast genomes. In the BGL-1 lines, one 7.8-kb hybridizing fragment was observed (FIG. 1H). Absence of the 4.0-kb wild-type fragment suggests that all the chloroplast genomes are transformed (to the detection limit of Southern blots) and therefore are homoplasmic.

### Example 3

### Transgene Segregation and Phenotype of BGL-1 Transplastomic Lines

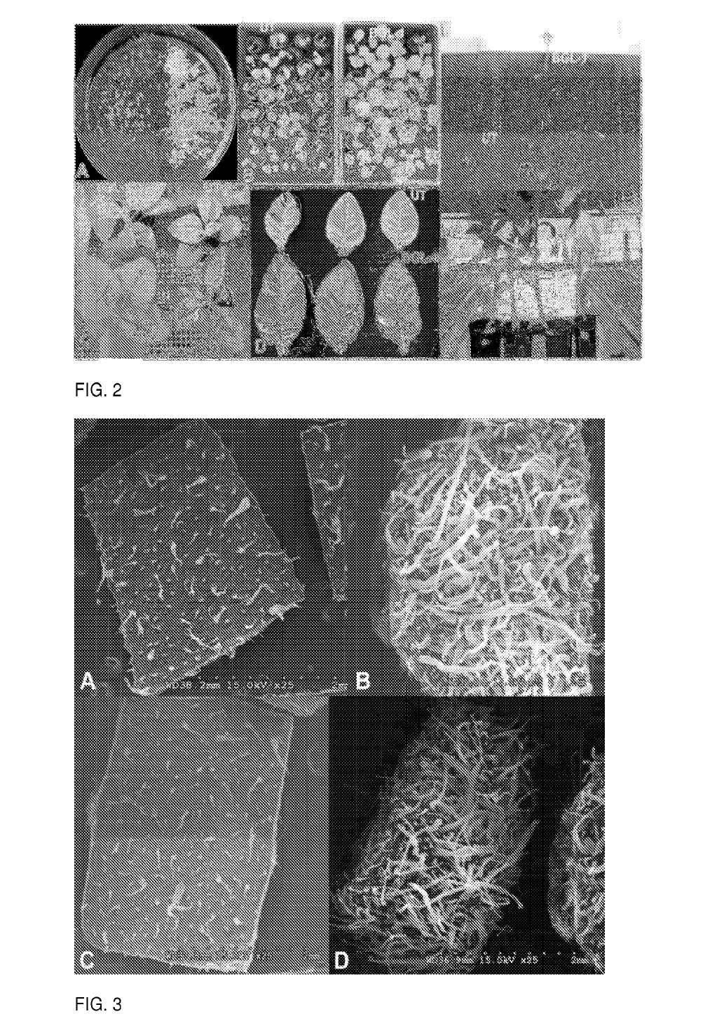

T1 transplastomic BGL-1 and UT seeds were germinated on half-strength MS medium containing spectinomycin (500 mg L⁻¹). Transplastomic BGL-1 seeds germinated and grew normally into green seedlings, whereas UT seedlings were bleached soon after germination (FIG. 2A). The lack of Mendelian segregation of transgenes in the BGL-1 line is evident in the progeny.

In order to investigate the phenotypes (plant height, internode length, flowering time, leaf area, biomass, etc.) of UT and transgenic lines, 12 transgenic plants from three independent transplastomic BGL-1 lines and 12 UT plants were grown in the greenhouse at 25° C., fertilized, and irrigated according to standard procedures. FIG. 2 (B and C) shows the same age of BGL-1 and UT seedlings, and significant differences in their size and height are evident. The average flowering time of BGL-1 plants was 1 month earlier than the UT control. The height of the mature BGL-1 line increased 150% when compared with the UT plants, because transplastomic plants have longer internodes (Table I). The leaf area of BGL-1 plants also increased by 160% (FIG. 2D); the average leaf area of BGL-1 was 780 cm² and that of UT was 490 cm² (Table I). The average biomass of the mature transplastomic plants was 190% higher than the UT line (Table I).

### Example 4

### High Levels of β-Glucosidase Activity in BGL-1 Leaves

Young, mature, and old leaves from transplastomic BGL-1 plants were collected, and β-glucosidase activity was measured using p-nitrophenyl β-d-glucopyranoside (pNPG) as the substrate. One unit of β-glucosidase is defined as the amount of enzyme that released 1 μmol of p-nitrophenol from pNPG substrate under the assay conditions described in “Materials and Methods.” β-Glucosidase activity (44.4 units g⁻¹ mature fresh leaves) was 160-fold more in transplastomic BGL-1 lines than in UT plants (0.27 units g⁻¹ mature fresh leaves). To calculate the yield of β-glucosidase enzyme in whole plants, leaves were collected and grouped into young, mature, and old. The yield of β-glucosidase enzyme was very high in transplastomic plants because the leaf biomass yield was almost 2-fold higher than in UT plants. Because 8,000 tobacco plants can be grown in 1 acre, it is possible to produce 130 million units per cutting, which is 390 million units per year (based on three cuttings; Table II).

### Example 5

### High Density of Trichomes in BGL-1 Leaves

Trichomes are specialized unicellular or multicellular structures derived from the epidermal cell layer and may have various functions depending on the plant species and organs (Wagner et al., 2004). Scanning electron microscopy analysis revealed two kinds of trichomes on both surfaces of tobacco leaves: glandular and nonglandular trichomes. The glandular trichomes differ in morphology and in the spectrum of compounds that are secreted. The glandular trichomes, with large heads, were found on both the lower and upper leaf surfaces. Trichome density was 10 times higher on the upper surface (FIG. 3, A and B) and 7.4 times higher on the lower surface (FIG. 3, C and D) of transplastomic BGL-1 lines when compared with UT controls (Table I).

### Example 6

### GA Hormone Levels are Elevated in BGL-1 Leaves

Endogenous GA levels were investigated because many of the phenotypical changes that occurred in the transplastomic line are known to be regulated by GA. Glucosyl conjugates of GAs are common endogenously occurring metabolites. They are expected to play a significant role in the regulation of the active hormone level (homeostasis) as well as in the processes of transport and storage (Schneider and Schliemann, 1994; Sembdner et al., 1994). The metabolic formation of GA conjugates has been described (Sembdner et al., 1994; Schneider et al., 2000). The reconversion of both GA-O-glycosides and GA glucosyl esters to free GAs has also been observed (Rood et al., 1983, 1986; Schneider and Schliemann, 1994). The metabolism of intact GA-O-glucosides, however, has not yet been detected. GA conjugates may play an important role in the control of growth and development of higher plants. It has been suggested that GA glucosyl esters are “deactivated” GAs that can be enzymatically reconverted to active GAs, thus serving as a reserve form of biologically active GAs. Two parallel GA biosynthetic pathways occur, the “nonhydroxylated” and the 13-hydoxylated pathways (Pimenta Lange and Lange, 2006), the latter one of which has been identified to be the major one in tobacco, and this was confirmed in this study for both BGL-1 and UT plants (Jordan et al., 1995; Table III). Therefore, we investigated GA hormones, their precursors, and metabolites of both pathways in UT control plants and BGL-1 lines (Table III).

Highest GAs levels were detected in leaves (Table III). There is a 2-fold increase in levels of GA precursor (GA₅₃, GA₄₄, GA₁₉, and GA₂₀), hormone (GA₁), and catabolite (GA₈) in the BGL-1 line when compared with UT controls (Table III). Therefore, the major GA pathway (13-hydroxylated pathway) is up-regulated in leaves of transplastomic lines. In other plant organs, GA precursor levels were similar (inflorescence) or even lower (shoot tip and internodes) in transplastomic lines when compared with UT plants. When compared with leaves in these organs, only low GA hormone levels were detectable, and they were similar (inflorescence) or even lower (shoot tip and internodes) in transplastomic lines than in UT plants.

In addition, the nonhydroxylated pathway was analyzed: GA₁₂, GA₁₅, GA₂₄, and GA₉ precursor levels (data not shown) and GA₃₄ catabolite levels (Table III) were more than 5 times lower compared with the respective GAs of the 13-hydroxylated pathway, confirming the latter one as the major pathway. However, GA₄ hormone levels were higher in leaves of transplastomic lines than of the UT plant (Table III). Taken together, an increase in active GA hormone levels is observed only in leaves where chloroplasts are abundant and β-glucosidase is expressed. All GAs analyzed form Glc conjugates, as they contain COOH (ester) and OH (ether) groups. Precursors can form conjugates, as they have COOH and OH side groups within the molecule. Therefore, release of active hormones from conjugates of precursors and mature forms by β-glucosidase is anticipated based on our hypothesis.

### Example 7

### IAA and Trans-Zeatin, but not ABA, Levels are Higher in BGL-1 Lines

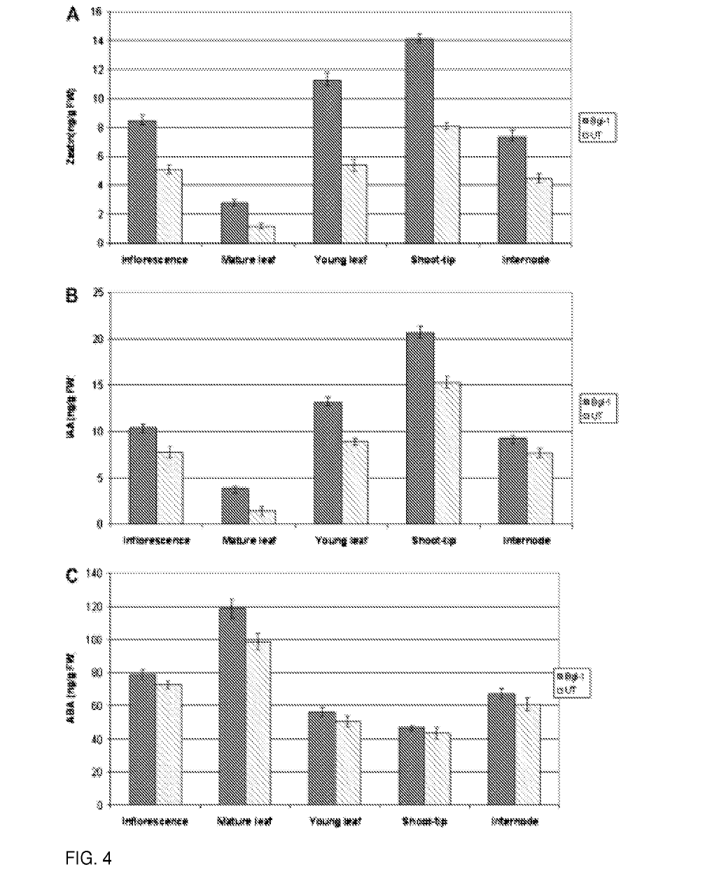

Several hormones form Glc conjugates, including ABA, zeatin, and IAA (Sembdner et al., 1994). Also, some of the phenotypes observed in BGL-1 lines (e.g., large leaf size) could be due to the action of other plant hormone groups. Therefore, all hormones or conjugates that we had the ability to evaluate and quantify were investigated using ELISA kits. In BGL-1 samples, the levels of IAA increased in all the tissues or organs. There was 130%, 140%, and 120% more IAA in inflorescence, shoot tip, and internode of BGL-1 than in the UT control. However, the IAA levels increased by 280% in mature leaves of BGL-1 lines (FIG. 4B), where more chloroplasts are present.

The trans-zeatin levels in BGL-1 samples also increased significantly. When compared with UT controls, trans-zeatin levels in inflorescence, shoot tip, and internode increased by 170%, 170%, and 160%, respectively. Notably, there were 230% and 210% higher trans-zeatin levels in mature and young leaves of BGL-1 than in the UT control (FIG. 4A), where more chloroplasts are present. In contrast, there was no significant increase of ABA levels in the BGL-1 samples when compared with the UT control, even in the young and mature leaves of the transplastomic BGL-1 lines (FIG. 4C), supporting the idea that ABA conjugates may be irreversible (Sembdner et al., 1994).

### Example 8

### Protoplast Culture without Exogenous Hormones or with Hormone Conjugates

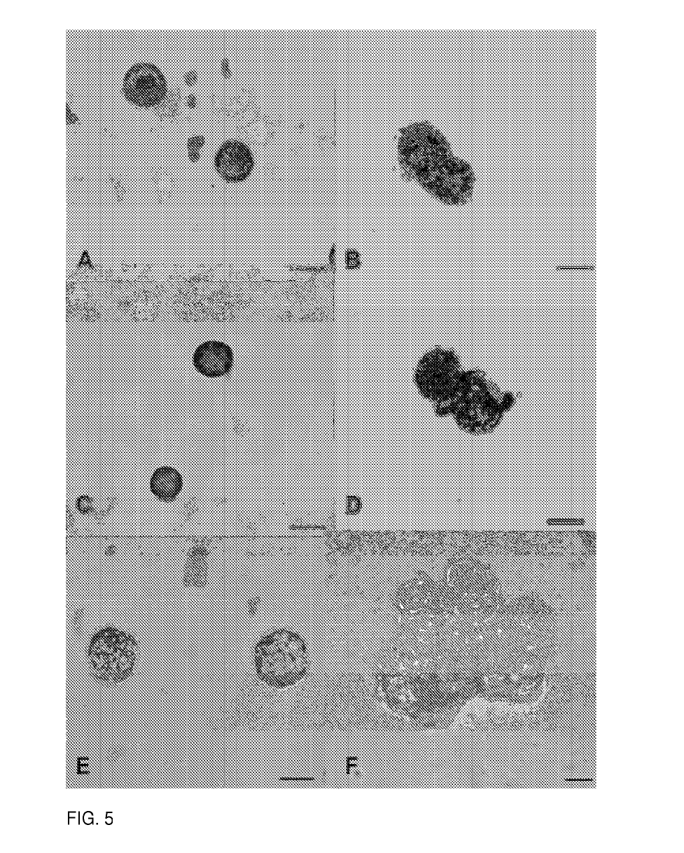

The enzyme cocktail with 2% (w/v) cellulase and 0.5% macerozyme could digest the leaf samples completely and release intact protoplasts. The yield of protoplasts from BGL-1 and the UT control was around 4 to 5×10⁶ g fresh weight, comparable to a previous report (Rao and Prakash, 1995). To evaluate the effects of hormones or hormone conjugates on protoplast division, six hormones or conjugate combinations (Table IV) were tested in the protoplast culture. Protoplasts from UT leaves did not divide and form cell colonies in the culture medium without exogenous hormone or when supplied with zeatin-O-glucoside only (FIG. 5, A, C, and E; Table IV). In contrast, protoplasts from BGL-1 leaves divided continuously (FIG. 5, B and D) and developed into cell colonies (FIG. 5F) in different types of culture even in the absence of added hormones (Table IV). Compared with the protoplasts from UT, the protoplasts from BGL-1 showed higher division and plating efficiency (Table IV). Most importantly, protoplasts from the BGL-1 line utilized exogenously supplied zeatin-O-glucoside, increasing the efficiency of their cell division by 670% when compared with UT protoplasts (Table IV).

### Example 9

### High Accumulation of Sugar Ester in BGL-1 Glandular Trichomes

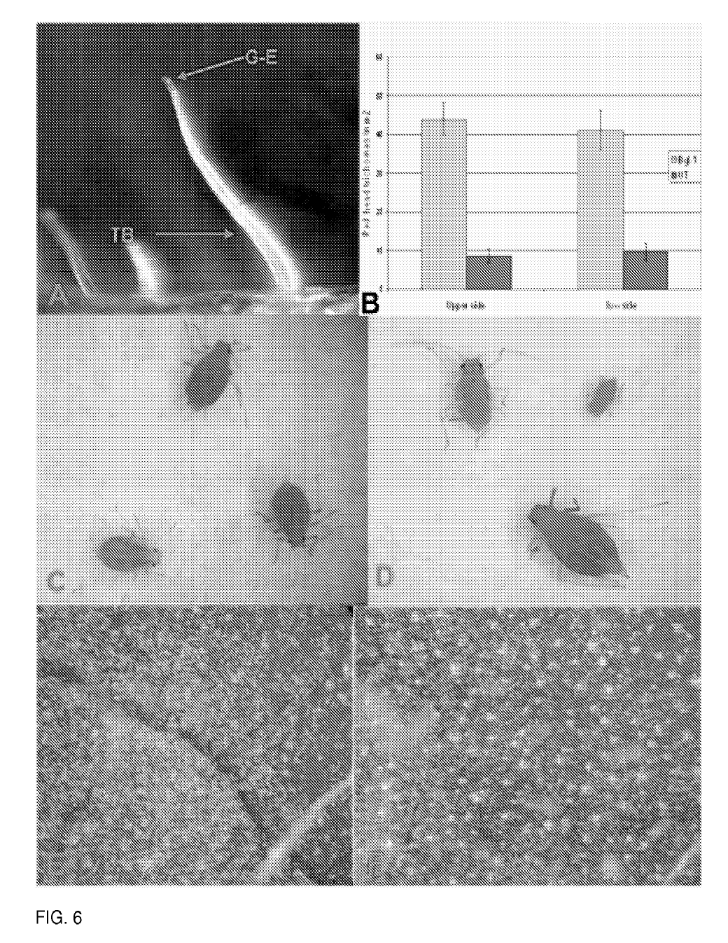

Natural sugar esters have been shown to be effective biopesticides against a range of insect species. Soft-bodied arthropods, including mites, lepidopteran larvae, aphids, whiteflies, and psyllids, are killed rapidly upon contact. In addition, sugar esters have demonstrated ovipositional and feeding deterrence against mites, whiteflies, and leaf miners (McKenzie et al., 2005). It has been well documented that the trichome gland is the only site of exudate sugar ester synthesis in tobacco (Severson et al., 1984), and our light microscope observations confirmed this. Tissues with glandular trichomes were stained by 0.2% rhodamine B, and only the trichome gland was stained (FIG. 6A). Aphids were placed on rhodamine B-stained leaf segments on glass slides and allowed to walk on the surface for 30 min. Slides were then placed in a chloroform atmosphere to anesthetize insects and then mounted for photography. It was observed that aphids walking on BGL-1 leaves were extensively contaminated with sugar ester (FIG. 6D), while the stain of wild-type aphids was very faint (FIG. 6C). It is well known that sugar ester, 4,8,13-duvatriene-1,3-diol, and labdanoids are predominant biopesticides excreted by tobacco glandular trichomes (Severson et al., 1984). This procedure was specific for sugar ester (4,8,13-duvatriene-1,3-diol and labdanoids are not stained) and can be used as a quantitative measure of sugar ester (Severson et al., 1984; Lin and Wagner, 1994). FIG. 6 (E and F) revealed that there are 4 to 5 times more glandular trichomes with red secretory heads from both BGL-1 leaf surfaces than on the UT leaves (FIG. 6B), suggesting that transplastomic plants produced more sugar ester than UT plants.

### Example 10

### BGL-1 Lines Confer Protection Against Whiteflies and Aphids

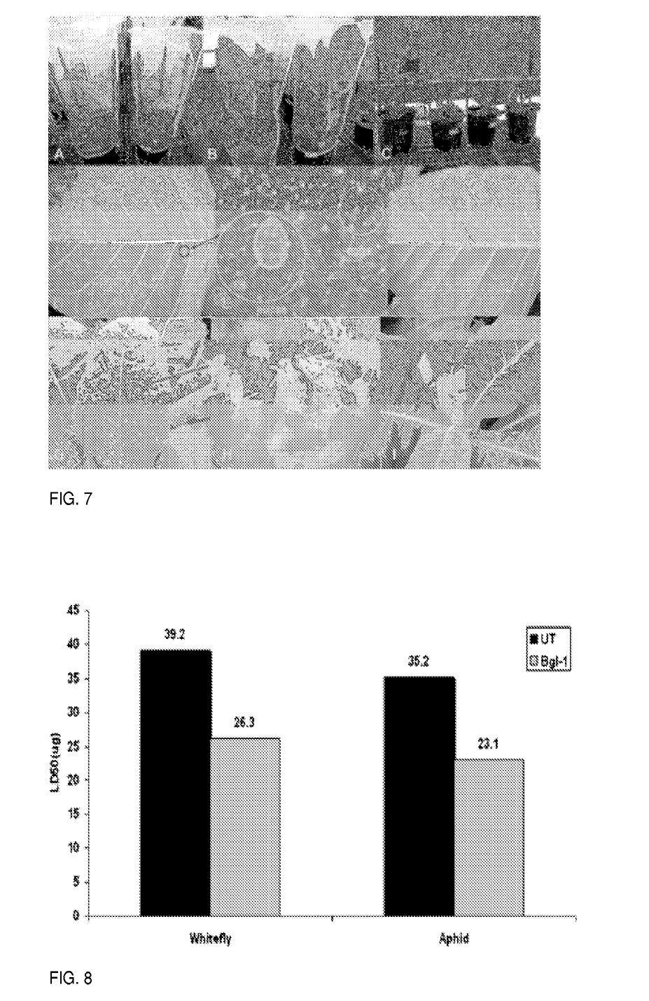

Both BGL-1 (from two independent lines) and UT samples for aphid and whitefly colonization tests included 10 plants each. Colonization tests of whitefly and aphid were performed in the greenhouse. Transplastomic and UT plants were covered with mesh bags in which 30 aphids or whiteflies were released (FIG. 7, A and B). The population buildup was recorded 25 d later (FIG. 7C). Significant differences in oviposition and the immature and adult populations were observed when whiteflies or aphids were released on control or transplastomic plants (Table V). The total number of whiteflies (eggs/pupae/adults) on the UT plants was 18-fold higher than on the BGL-1 transplastomic lines. FIG. 7 (D and E) show a large population of whiteflies on the UT leaves, whereas only a few of whiteflies were found on the transplastomic plants (FIG. 7F). Similarly, heavy colonization of aphids on the control plants was very apparent (FIG. 7, G and H) when compared with transplastomic plants (FIG. 7I). The size of the aphid population on the UT plants was 15 times more than on the BGL-1 transplastomic lines (Table V).

In addition to colonization tests, the toxicity of exudates was also evaluated using the method reported by Jackson and Danehower (1996) and Wang et al. (2001). Purified exudates from tobacco leaf surface were applied as droplets of 0.2 μL size in a solvent of Suc acetate isobutyrate:acetone (2:1, v/v) to dissolve exudates. This solvent alone caused no mortality over the 66-h time period of toxicity tests (Wang et al., 2001). According to Spearman-Karber method dose-response analysis of whitefly to total exudates, LD₅₀ values (lethal dose to kill 50% of the test population) of 26.3 and 39.2 μg per whitefly for BGL-1 and UT were obtained. Similarly, the LD₅₀ values for aphid were 23.1 and 35.2 μg per aphid for BGL-1 and UT exudates (FIG. 8). These results are very similar to the results reported by Wang et al. (2001), in which genetically modified plants showed a LD₅₀ value of 20.8 when compared with 32.2 in UT plants.

Materials and Methods for Examples 1-10

Isolation of the bgl1 Gene and Construction of the Chloroplast Vector.

Full-length cDNA of bgl1 (U09580) was amplified using overlapping primers for three exons and genomic DNA of Trichoderma reesei (American Type Culture Collection) as the template by a PCR-based method (An et al., 2007). The forward primers for three exons are as follows: 5′-GAATTCCATATGCGTTACCGAACAGCAGCTGCGCTGGCACTTGCCACTGGGCCCTTTGCTAGGGCAGACAGTCACTCAACATCGGGGGCC-3′ (exon 1); 5′-CTAGGGCAGACAGTCACTCAACATCGGGGGCCTCGGCTG-3′ (exon 2); and 5′-CACGCCGCGGTACGAGTTCGGCTATGGACTGTCTTACACCAAGTTCAACTACTCACGCC-3′ (exon 3). The reverse primers for exons 2 and 3 are as follows: 5′-ACAGTCCATAGCCGAACTCGTACCG-3′ (exon 2); and 5′-CTCTCTAGACTACGCTACCGACAGAGTGCTCGTC-3′ (exon 3). Sequences for restriction enzymes NdeI and XbaI were added in forward and reverse primers to facilitate cloning into the pLD vector. Full-length amplified bgl1 was ligated into the pCR Blunt II Topo vector (Invitrogen) and sequenced (Genewiz) to detect any PCR errors. The bgl1 gene was released from the Topo vector by digestion with NdeI and XbaI and cloned into the pLD vector (Daniell et al., 2001; Verma et al., 2010b) to make the tobacco (Nicotiana tabacum) chloroplast expression vector. All cloning steps were carried out in Escherichia coli according to Sambrook and Russell (2001).

Bombardment and Selection of Transplastomic Lines.

Tobacco leaves were bombarded using the Bio-Rad PDS 1000/He biolistic device as described previously (Daniell et al., 2004). Bombarded leaves were then subjected to three rounds of selection. First and second rounds of selection were performed on the regeneration medium of plants, and the third round of selection was on hormone-free half-strength MS medium. All growth media were supplemented with 500 mg L⁻¹ spectinomycin as described previously (Verma et al., 2008). After selection, confirmed transplastomic lines were transferred to the pots in the greenhouse for further growth.

PCR Evaluation of Transplastomic Lines.

Total plant DNA was isolated from UT and transplastomic tobacco leaves using the DNeasy Plant Mini Kit (Qiagen). PCR was set up with two pairs of primers, 3P-3M and 5P-2M (Verma et al., 2008), to investigate the integration of transgene expression cassettes into the tobacco chloroplast genome. The 3P primer (5′-AAAACCCGTCCTCAGTTCGGATTGC-3′) anneals with the native chloroplast genome in the 16S rRNA gene, while 3M primer (5′-CCGCGTTGTTTCATCAAGCCTTACG-3′) anneals with the aadA gene. This pair of primers was used to investigate site-specific integration of selectable marker genes into the chloroplast genome. The 5P primer (5′-CTGTAGAAGTCACCATTGTTGTGC-3′) anneals with the aadA gene, while the 2M primer (5′-TGACTGCCCACCTGAGAGCGGACA-3′) anneals with the trnA gene, which was used to confirm the integration of the transgene expression cassette.

Confirmation of Homoplasmy and Transgene Segregation.

Southern-blot analysis was performed to evaluate homoplasmy according to laboratory protocols (Kumar and Daniell, 2004). In brief, total tobacco genomic DNA (2-4 μg) isolated from UT or transformed leaves after the third round of selection was digested with SmaI and separated on a 0.8% agarose gel and then transferred to a nylon membrane (Nytranspc; Whatman). The chloroplast flanking sequence probe was prepared by digesting pUC-Ct vector (Verma et al., 2008) DNA with BamHI and BglII, which generated a 0.81-kb probe (FIG. 1A). After labeling the probe with [α-³²P]dCTP, the membrane was hybridized with the probe using the Stratagene Quick-Hyb hybridization solution and protocol.

T1 seeds from transplastomic line BGL-1 and UT tobacco seeds were geminated on half-strength MS medium containing 500 mg L⁻¹ spectinomycin for the evaluation of segregation of transgenes.

Phenotypic Evaluation of Transplastomic Lines.

In order to investigate the phenotypes (plant height, internode length, flowering date, leaf area, biomass, etc.) of UT and transgenic lines, 12 transgenic plants from three independent transplastomic BGL-1 lines and 12 UT plants were transferred to jiffy pellets, kept initially for 2 weeks under high humidity, and then moved to the greenhouse for further growth at 25° C., fertilized, and irrigated according to standard procedures.

Scanning Electron Microscopic Evaluation of Leaf Surface and Histochemical Staining of Sugar Ester.

Leaves were washed with distilled water to remove any dirt and dead bodies of insects. A drop of fixative (2.5% glutaraldehyde and 2% paraformaldehyde in 0.1 m phosphate buffer) was added in a petri dish or on a glass plate. Small pieces were dissected (3-4 mm) from mature leaves of transplastomic and UT plants in the presence of the fixative. At room pressure, the specimens sunk to the bottom. Tissues were fixed for 3 to 4 h at room temperature. Tissues were washed with 0.1 m phosphate buffer four times for 15 min each and then rinsed with distilled water three times for 5 min each. Tissues were dehydrated with a graded series of ethanol: 30% ethanol for 10 min; 50% to 70% to 80% to 90% to 95% ethanol for 20 min each; and finally, 100% ethanol for 20 min three times. Leaf cross-sections were loaded into a gasket and placed into the critical point drier (Bomb; Electron Microscopy Sciences). After drying, samples were placed on carbon strips facing up. Gold-Palladium was coated with the Emitech K 550 Sputter Coater for 2 min to reach 200 Å. Pieces were excised and examined with a Hitachi S-3500N scanning electron microscope. The densities of trichomes were determined on both the upper and lower sides of the leaves.

For sugar ester staining, tissue pieces were submerged in 0.2% rhodamine B in water for 60 min, then submerged in four separate vessels containing distilled water (5 s in each) to remove unbound stain. Samples were photographed using a Stemi V6 stereomicroscope.

Evaluation of Aphid and Whitefly Toxicity.

Exudates were washed from five to seven developmentally matched leaves of healthy BGL-1 and UT plants by acetonitrile using a protocol essentially as described by Wang et al. (2001). Washes were evaporated, and exudates were dried and weighed. Dilutions were prepared in 2:1 (v/v) Suc acetate isobutyrate:acetone. In three of the four experiments, doses were 5, 12.5, 25, and 50 μg. In the fourth experiment, doses of 6, 10, 20, 38, and 50 μg were used. Mature aphids (Myzus persicae) and whiteflies (Bemisia tabaci) were collected from greenhouse-grown UT tobacco plants and distributed among seven leaf discs (1.5 cm diameter) on 2% agar on petri plates (20 aphids per plate, one plate per dose). The discs were cut from leaves washed with 20% (v/v) Tween 20 to remove exudate and then with water. One drop of exudate-containing solution (0.2 μL) was applied to dorsa of each aphid (20 aphids or whiteflies per dose per experiment). After 66 h at 22° C. and a 16-h-light/8-h-dark cycle, mortality was assessed. The LD₅₀ values for 66 h were estimated according to the Karber method (Zhang et al., 2004).

Evaluation of Aphid and Whitefly Colonization.

BGL1-1 and UT plants were challenged by aphids using the Yao et al. (2003) protocol and by whiteflies using the Jindal and Dhaliwal (2009) protocol. Plants were grown inside the greenhouse, fertilized, and irrigated according to standard procedures. During the bioassay, the whole plant (40 d old, six- to seven-leaf stage) was confined to an insect-proof nylon mesh bag and maintained at 25° C. for 25 d. This system allowed aphids access to the entire plant but confined them to a single plant. Thirty neonatal nymphs were introduced with a hair brush to each tobacco plant on day 0. For the whitefly bioassay, 30 newly emerged adults were released in mesh bags as described above. The mesh bags were removed after 25 d, and the total number of adult and immature insects that emerged was recorded on the whole plant. Both BGL-1 (from two independent lines) and UT samples for aphid and whitefly colonization tests included 10 plants each.

β-Glucosidase Enzyme Assay

Total soluble protein was extracted from 1 g of fresh leaf tissue ground in liquid nitrogen using 2 mL of ice-cold 100 mm citrate buffer (pH 5.2) with the protease inhibitor cocktail (Roche). Protein concentration was measured according to the Bradford method using the Bio-Rad protein assay kit. β-Glucosidase activity was estimated by the Berghem and Pettersson (1974) method with the following modifications: 100 μg of total soluble protein extracted from leaf sample was incubated with 4 mm pNPG (Sigma) in 1 mL of 100 mm citrate buffer, pH 5.2, at 50° C. for 10 min. The reaction was terminated by adding 2 mL of 1 m Na₂CO₃. Enzymatic release of nitrophenol was spectrophometrically determined immediately (after adding Na₂CO₃) at 405 nm. The standard curve of p-nitrophenol was prepared under alkaline conditions using 1 m Na₂CO₃. The concentration of nitrophenol present in the reaction was analyzed by measuring the absorbance and extrapolating the concentration from the nitrophenol standard curve. One pNPG unit is defined as 1 μmol of p-nitrophenol formed per min at 50° C. under these assay conditions.

Evaluation of GAs, Precursor, and Metabolites by Gas Chromatography-Mass Spectrometry

Five fully grown plants (with 14-16 leaves) from BGL-1 and UT were sampled for GA analysis. Five-gram fresh samples from mature leaves (seventh or eighth), inflorescence, shoot tip, internodes, and young leaves (top first or second) were ground into fine powder in liquid nitrogen and freeze dried in a lyophilizer. All the samples were stored at −20° C. until analysis. Freeze-dried plant material (200 mg dry weight) was spiked with 17,17-d2-GA standards (2 ng each; from Prof. L. Mander). Samples were extracted, purified, derivatized, and analyzed by combined gas chromatography-mass spectrometry using selected ion monitoring as described by Lange et al. (2005). Six successive GAs of the nonhydroxylated pathway (GA₁₂, GA₁₅, GA₂₄, GA₉, GA₄, and GA₃₄) and six of the 13-hydroxylated pathway (GA₅₃, GA₄₄, GA₁₉, GA₂₉, GA₁, and GA₈) were further analyzed.

ABA, IAA, and Zeatin Detection by ELISA

The ABA, IAA, and zeatin concentrations in shoot tip, inflorescence, internode, and mature and young leaves from BGL-1 and UT control plants were measured using the Phytodetek competitive ELISA kits (Agdia). The hormone extraction was done based on the Oliver et al. (2007) protocol. BGL-1 and control plants were grown in the greenhouse in the same conditions, and then shoot tip, inflorescence, internode, and mature and young leaves were collected at a fixed time of day, frozen, and ground in liquid nitrogen. The powder was extracted overnight at 4° C. in cold 80% methanol. The mixture was then centrifuged at 5,000 rpm for 5 min, and the supernatant was collected. The pellet was washed three times in cold 80% methanol. Then, the supernatant of all the samples was pooled and dried in a Speed-Vac until approximately 50 μL of liquid remained. Tris-buffered saline (25 mm Tris-HCl, pH 7.5, 100 mm NaCl, 1 mm MgCl₂, and 3 mm NaN₃) was added to a final volume of 200 μL. These extracts were diluted 10-fold in Tris-buffered saline and used in the ELISA according to the Phytodetek kit protocol. A standard curve of different ABA, IAA, and zeatin dilutions was constructed to calculate the sample ABA, IAA, and trans-zeatin concentrations. ABA, IAA, and trans-zeatin concentrations were calculated as ng per g fresh weight. Each measurement was replicated three to four times using pooled samples.

Protoplast Isolation and Culture

The protocol of the protoplast isolation was modified from the method of Rao and Prakash (1995). Leaves from BGL-1 and UT were cut into small pieces and placed on the surface of the enzyme solution with the lower surface down. Incubation was carried out in the dark at a temperature of 25° C.±1° C. for 8 h to release the protoplasts. The enzymatic cocktail contained 2% (w/v) cellulase Onozuka R-10, 0.5% (w/v) macerozyme R-10, 0.2% (w/v) dextran potassium sulfate, 5 mm CaCl₂.2H₂O, and 0.7 m mannitol, pH 5.8. After removing undigested materials and digested debris by filtration through a 400-m steel mesh, the enzymatic mixture was centrifuged at 1,000 rpm for 10 min. The protoplasts were resuspended in 5 mL of washing medium, and the process was repeated three times. Protoplasts were cultured at a density of 2×10⁵ mL⁻¹ in 5-cm petri dishes in 3 mL of culture medium (Rao and Prakash, 1995) supplemented with different hormone or conjugate hormone regimes (Table IV). The cultures were kept at 25° C. in the dark. After 7 d, fresh medium was added to each petri dish, and the addition of medium was repeated on the 11th d of culture. Star-shaped microcalli developed within 21 d of culture. After the development of microcalli visible to the naked eye, the cultures were transferred to light.

### Example 11

### Construction of Transformation Vectors for Vacuolar Targeting of β-Glucosidase

The bio-activating β-glucosidases in eudicotyledons are stored in the apoplast as an enzyme or protein bodies. In monocotyledons, β-glucosidases are localized in plastids or other compartments. Therefore, it has been realized that in order to release the most active hormones, β-glucosidase should be targeted to the vacuole or expressed in chloroplasts to investigate the mechanism of β-glucosidase to release phytohormones from inactive glucoside conjugates and transfer from this model system to economically important crops.

To target beta glucosidase (Bgl1) from Trichoderma reesei to vacuole, a C-terminal propeptide (CTPP) of Concanavalin A, Chitinase A and N-terminal propeptide (NTPP) of sporamin was used. Gene encoding beta glucosidase (bgl1) was isolated from Trichoderma reesei by PCR using sequence specific primers and genomic DNA as template. PCR product was cloned in pCR BluntII Topo vector (Invitrogen) and sequenced. To add different targeting sequences and his tag to bgl1 coding sequence, following primers were synthesized from Integrated DNA Technologies (IDT).

Using Topo vector containing bgl1 gene as template, first PCR was carried out with primer pairs DV1/DV2, DV1/DV4 and DV7/DV9. PCR products were gel purified, cloned in pCR BluntII Topo vector and the recombinant clone was used as template for next round of PCR. Topo vector harboring DV1/DV2 PCR product was used as template with primer pairs DV6/DV3 and DV1/DV3 resulting in His-Bgl1-ConA-CTPP and Bgl1-ConA-CTPP respectively. Topo vector harboring DV1/DV4 PCR product was used as template for primer pairs DV6/DV5 and DV1/DV5 resulting in His-Bgl1-Chitinase-CTPP and Bgl1-Chitinase-CTPP respectively. Topo vector harboring DV7/DV9 PCR product was used as template for primer pairs DV8/DV10 and DV8/DV9 resulting in Sporamin-NTPP-Bgl1-His and Sporamin-NTPP-Bgl1 respectively. All PCR products were cloned into the pCR BluntII Topo vector and sequenced. The bgl1 sequence with or without his tag and including vacuolar targeting sequences was excised (SnaBI/XbaI) from recombinant topo vectors and cloned into binary vector pCAMBIA 2300S (SmaI/XbalI): this placed the bgl1 sequence with vacuole targeting sequences downstream of the CaMV35S promoter and upstream of the nos polyA (FIG. 8). The CAMBIA 2300S vector also has plant selection gene nptII encoding resistance to kanamycin. The selection gene is driven by CaMV35S promoter and terminated by CaMV35S polyA. The resulting recombinant plasmid DNA was transformed into A. tumefaciens LBA4404 Rif^{R} competent cells using freeze-thaw method. Selection was done on media containing kanamycin (50 μg/ml) and rifampicin (100 μg/ml) at 30° C. Transformants were screened by PCR using primer pairs DV1/DV9. Agrobacterium tumefaciens harboring the recombinant plasmid was used for Agrobacterium-mediated transformation.

### Example 12

### Agrobacterium-Mediated Transformation of Tobacco and Artemisia Harboring β Glucosidase Gene with Different Vacuolar Targeting Sequences

Plant Material and Agrobacterium Culture:

Tobacco cultivar Petit Havana and Artemisia annua seeds were surface sterilized and germinated on ½ MS basal medium. Agrobacterium was grown overnight in YEP liquid medium containing rifamycin (50 mg/l) at 28° C. under agitation. Then, OD was adjusted to 0.1 at 600 nm.

Plant Transformation and Selection:

Tobacco and Artemisia leaf explants from 6 to 8 weeks old in vitro seedlings were cut into small pieces and inoculated with overnight grown Agrobacterium suspension cultures harboring Bgl1 gene with different vacuolar targeting sequences for 10-20 min. After inoculation, the explants were placed on RMOP (MS basal supplemented with 100 mg/l myoinositol, 1 mg/l thiamine HCl, 1 mg/l BAP, 0.1 mg/L NAA, 30 g/l sucrose, pH 5.8, 5 g/l phytoblend) and ARM (Artemisia regeneration medium; MS basal medium supplemented with 100 mg/l myoinositol, 1 mg/L BAP, 0.05 mg/L NAA, 30 g/L sucrose, pH 6.1, 5 g/l phytoblend) and co-cultured under dark condition at 25° C. for 2-3 days. Following co-culture, the explants were washed with sterile water for 3-4 times until no bacterial turbidity was observed. Then, the explants were subjected to a final wash with water containing cefotaxime (400 mg/l) to kill the bacteria and dried on a filter paper. The dried explants were then transferred to RMOP and ARM medium containing kanamycin (50 mg/l for tobacco and 30 mg/l for Artemisia) supplemented with cefotaxime (400 mg/l). The explants were cultured in vitro under 16 hour photoperiod. The regenerated shoots on selection medium were transferred to ½ MS basal salt supplemented with 20 g/l sucrose, pH 5.8 and 4 g/l phytoblend containing kanamycin and cefotaxime to induce rooting.

Molecular Characterization:

Total genomic DNA was extracted from tobacco and Artemisia putative transformants. Integration of Bgl1 gene was confirmed by PCR using Bgl1 gene specific primers. In order to check Bgl1 transcript level, total RNA was extracted from PCR positive tobacco and Artemisia transgenic lines and performed Northern blot with Bgl1 probe. All the PCR and Northern positive transgenic plants were transferred to green house.

Bgl1 Enzyme Assay:

β-glucosidase enzyme activity for tobacco transgenic plant (one with highest Bgl1 transcript level) was quantitatively determined by using a pNPG spectrophotometric assay. In brief, total protein was extracted from untransformed and tobacco transgenic plants and 100 μg of total soluble protein were incubated with 4 mM of substrate p-nitrophenyl-β-D-glucopyranoside (pNPG) in 0.1 M citrate buffer at different pH condition. The reaction was incubated at 50° C. for 120 minutes and the reaction was stop by adding 2 ml of 1M Na₂CO₃ and absorbance was read at 405 nm. The activity was based on μM of pNP release from pNPG. The pNP standards were prepared at different concentrations and a standard graph was plotted. The activity was determined by comparing the absorbance in the assay with the standard graph.

Scanning Electron Microscopy:

Tobacco and Artemisia leaf samples from untransformed and transgenic lines were collected and washed with distilled water to remove dirt. The leaves were cut into small pieces and placed in a tube containing 1 ml of fixative solution containing 2.5% glutaraldehyde, 2% paraformaldehyde in 100 mM phosphate buffer and fixed at 4° C. overnight. The leaves were washed for 3 times with 100 mM phosphate buffer and then dehydrated with ethanol. Following serial dehydration with ethanol, leaf samples were then dried by critical point drying and then mounted on carbon strips. Finally, these samples were coated with Gold and Palladium in a sputter coater for 2 minutes. Then the samples were analyzed in scanning electron microscope.

Results

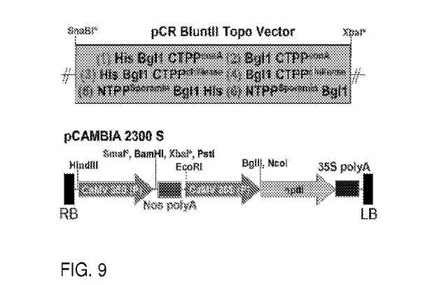

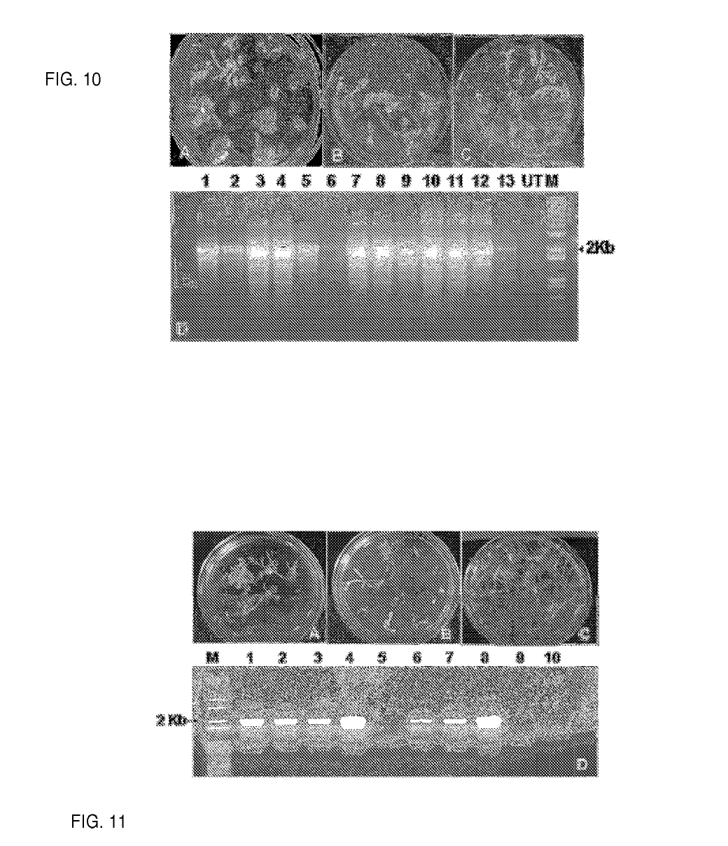

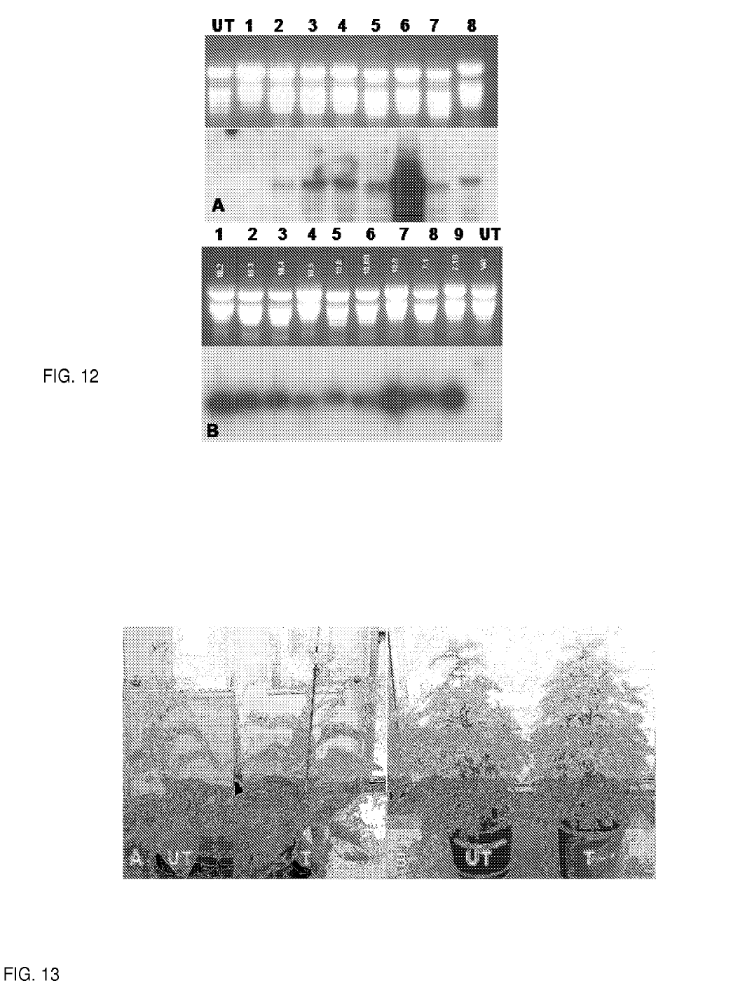

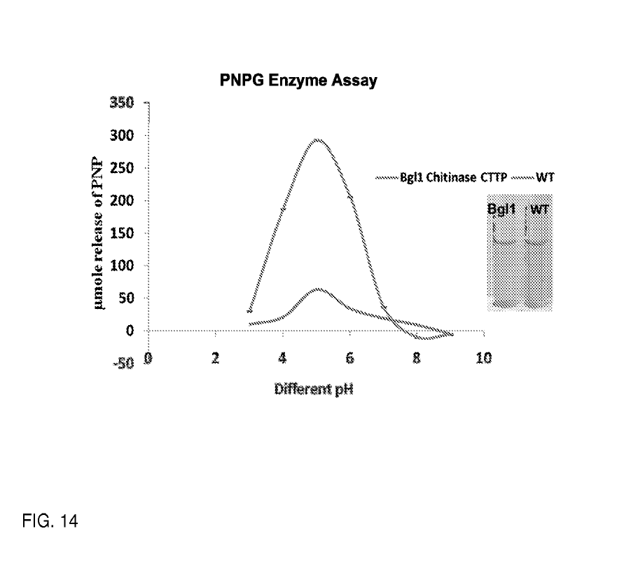

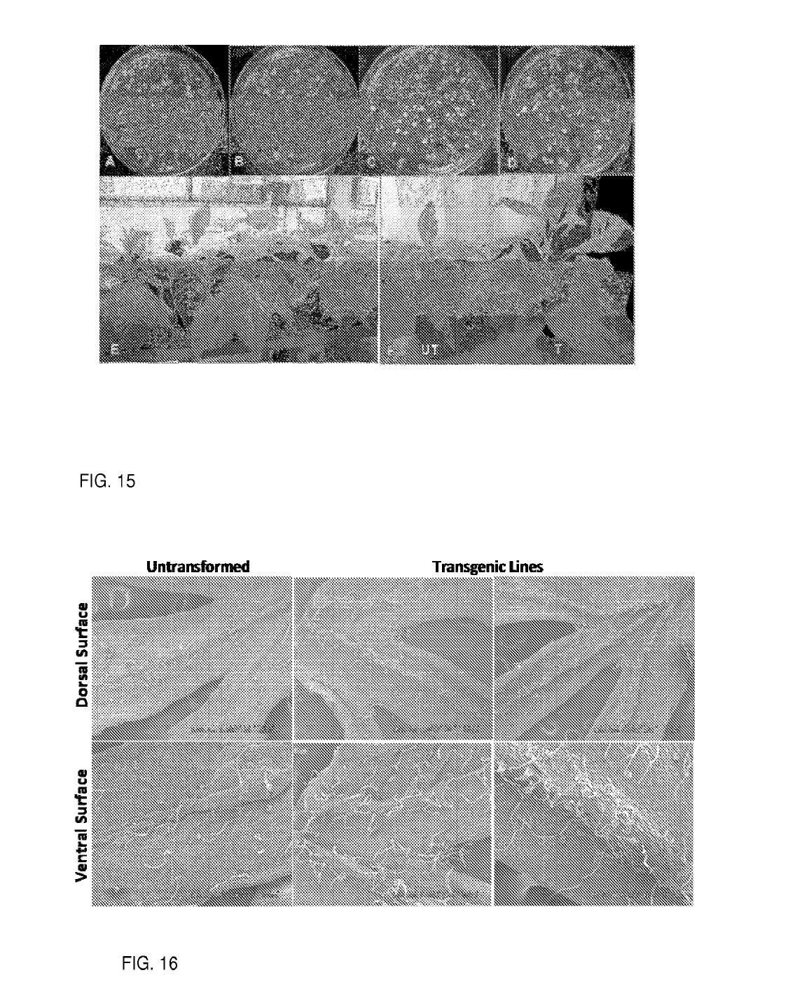

Many putative transformants were regenerated from tobacco and Artemisia leaf explants after 4 to 5 weeks of culture on regeneration medium containing kanamycin and cefotaxime. Genomic DNA was extracted from untransformed and putative transformants. The integration of Bgl1 gene was confirmed by PCR using Bgl1 gene specific primers and showed an amplification product which was absent in untransformed plants (FIGS. 9 & 10). All the PCR positive plants were selected and transferred to jiffy palettes. RNA was extracted from the PCR positive plants and Bgl1 transcript level was checked by Northern blot analysis using Bgl1 gene specific probe. Many Bgl1 transcript positive transgenic lines were obtained from tobacco and Artemisia plant with different transcript level (FIG. 11). All the transgenic plants were transferred to green house and their phenotype were compared with untransformed plants. In T0 plants, no significant difference in phenotype was observed in between untransformed and transgenic lines (FIG. 12). In order to check Bgl1 enzyme activity, tobacco transgenic line with highest transcript level was selected and β-glucosidase enzyme activity for was quantitatively determined by using a pNPG spectrophotometric assay at different pH condition. Among the different pH ranges, highest enzyme activity was observed at pH 5. Transgenic line showed 4 folds enzyme activity than untransformed plant (FIG. 13). Seeds from T0 tobacco transgenic lines were collected and germinated on ½ MS basal medium containing kanamycin. Different pattern of Mendelian segregation was observed in different ratios (FIG. 14A-D). All the green and healthy seedlings were selected and transferred to green house. In T1 tobacco plant, transgenic plants showed little more biomass than untransformed (FIG. 14F). Detail studies of the biomass comparison will be performed in homozygous transgenic lines. It has been reported that the trichomes are directly involved in insect resistant in tobacco and artemisinin production in Artemisia. The differences in leaf trichomes density and morphology of untransformed and Bgl1 transgenic lines were analyzed by scanning electron microscope. There is significant increase in trichome density in both the upper and lower leaf surfaces of Artemisia annua Bgl1 transgenic lines when compared to untransformed control, potentially increasing the content of artenisinin FIG. 15).

### Example 13

### β-Glucosidase Expression in Lettuce

Agrobacterium Mediated Transformation and Selection of Lettuce (Var Simpson Elite):

The lettuce wild type leaves were cut into small explants and suspended in water to keep them hydrated. Then the explants were co-cultivated with different clones of Agrobacterium tumefaciens in lettuce regeneration liquid medium for 10-20 min. After that the explants were dried on a filter paper and then placed on lettuce regeneration agar medium. The agar plates containing the explants were incubated in dark for 2-3 days. Then the explants were washed with water for 3-4 times until no bacterial turbidity is observed. This is done in order to remove the Agrobacterium. Then the explants were subjected to a final wash with water containing antibiotic cefotaxime (400 mg/l) to kill the bacteria. After that the explants were dried on a filter paper and then transferred to selection medium containing antibiotics, Kanamycin (50 mg/l) as the selection agent and Cefotaxime (400 mg/l) as an antimicrobial. The explants were kept in a 16 hour photoperiod environment invitro in order to generate regenerated shoots. Once the regenerated shoots were obtained, they were transferred to ½ MSO agar media amended with Kanamycin (50 mg/l) for root induction.

PCR Analysis for Confirmation of Transgene Integration:

Genomic DNA was extracted form untransformed and transgenic lines. The extracted DNA was then subjected to PCR analysis in order to screen putative transformant shoots for transgene integration. A 25 μl PCR reaction was set up using forward and reverse primers corresponding to the selection marker gene Neomycin phosphotransferase (nptII). Then the reaction was amplified in a thermocycler (Bio-rad PTC-100 peltier thermo cycler). The reaction for 1 cycle was Denaturation at 95° C. for 5 min followed by another step of 95° C. for 1 min, Annealing at 56° C. for 1 min, primer extension at 72° C. for 1 min. The entire reaction cycle was set to 30 cycles followed by a 10 min final extension at 72° C. After amplification the samples were run on a 0.8% agarose gel and then examined in a gel documentation system (Bio-rad). The samples were compared with an wild type DNA which was used as an untransformed negative control.

Southern Blot Analysis of the PCR Positive Plants:

After confirming amplification by PCR, the shoots were then screened for determining gene integration and copy number by southern blot analysis. Genomic DNA was digested with HindIII restriction enzyme overnight. After that the digested samples were separated on a 0.8% agarose gel overnight at 20V. Then the gel was transferred to nylon membrane and hybridized with β-glucosidase gene specific probe following lab protocol.

Northern Blot Analysis for Detecting β-Glucosidase Transcripts:

The transgenic lines were then analyzed for presence of gene specific transcript by northern blot. RNA was extracted from different plant samples using the iNtRON RNA extraction kit. The extracted RNA was then quantified in a nanodrop spectrophotometer. Then 10-20 μg of RNA was taken and then run on a gel containing 1× MOPS and equal volume of formaldehyde. The RNA was then transferred overnight to a nylon membrane and hybridization was performed as per established protocol using a β-glucosidase gene specific probe.

Gel Diffusion Assay:

In order to visualize the activity of β-glucosidase, a gel diffusion assay was performed using a fluorescent substrate 4-methylumbelliferyl-β-D-glucopyranoside (4-MUG). The substrate was dissolved in an agarose solution prepared in 0.1 M citrate buffer at pH 5.0. Then the substrate solution was cast on a square petriplate and then impregnated with wells. Then wild type and transgenic plant protein extracts of concentrations ranging from 100-200 μg were added and then incubated overnight at 37° C. Then the gel plate was then placed on a UV transilluminator for visualizing halos which represent enzyme activity.

Enzyme Assay:

β-glucosidase enzyme activity was quantitatively determined by using a pNPG spectrophotometric assay. Protein was extracted from untransformed and Bgl1 transgenic lines. A total of 100 μg of total soluble protein was incubated with 4 mM of substrate p-nitrophenyl-β-D-glucopyranoside (pNPG) in 0.1 M citrate buffer at pH 5.2. The reaction was incubated at 50° C. for 120 minutes. Then 2 ml of 1M Na₂CO₃ was added to the reaction and absorbance was read at 405 nm. The activity was based on μM of pNP release from pNPG. The pNP standards were prepared at different concentrations and a standard graph was plotted. The activity was determined by comparing the absorbances in the assay with the standard graph.

Optimization of pH:

The optimum pH of β-glucosidase was determined by performing the pNPG assay at different pH levels. Different buffers were used for varying pH values, 0.1 M citrate buffer pH 3-5, 0.1 M Tris buffer pH 6 & 7, 0.1M phosphate buffer pH 8 & 9. 100 μg of enzyme was incubated with 4 mM of pNPG substrate and incubated for 120 minutes. Then absorbance was read at 405 nm after adding 2 ml of 1M Na₂CO₃.

Optimization of Temperature:

Once pH optimum was determined, that specific pH was used to setup the temperature optimization reactions. 100 μg of protein was incubated with 4 mM of pNPG substrate at different temperatures (25° C., 37° C., 50° C., 60° C. & 70° C.). The reaction was set for 120 minutes and then 2 ml of 1M Na₂CO₃ was added. Then the absorbance was read at 405 nm.

Optimization of Substrate Concentration:

The optimum substrate concentration was determined by incubating the enzyme at different substrate concentrations. 100 μg of enzyme was incubated with varying concentrations of pNPG substrate (2-16 mM of substrate) under optimum pH and temperature. The reaction was set for 120 minutes and then absorbance was read at 405 nm following addition of 2 ml of 1M Na₂CO₃.

Leaf Sample Preparation for Scanning Electron Microscopy:

Both wild type and transgenic lettuce leaves were taken and washed with distilled water to remove dirt, insects etc. The leaves were cut into small pieces and placed in eppendorf tubes containing 1 ml of fixation solution containing 2.5% glutaraldehyde, 2% paraformaldehyde in 100 mM phosphate buffer. Then the tubes were kept open and placed in vacuum for 20 minutes until the air bubbles are gone. Then the tubes were stored at 4° C. overnight. The next day the fixation solution was removed and the leaves were washed for 3 times with 100 mM phosphate buffer. The leaves were then dehydrated with graded series ethanol 9 ranging from 30%-100%). The leaves were treated with 30% ethanol for 10 min followed by 50,70,80,90 & 95% each for 20 minutes. Finally the leaves were then treated with 100% ethanol for 20 min thrice. After that the leaves were then dried by critical point drying and then placed on carbon strips. Finally the leaves were then coated with Gold and Palladium in a sputter coater for 2 minutes. Then the leaves were analysed in scanning electron microscope.

Results

Confirmation of Transgene Integration by PCR:

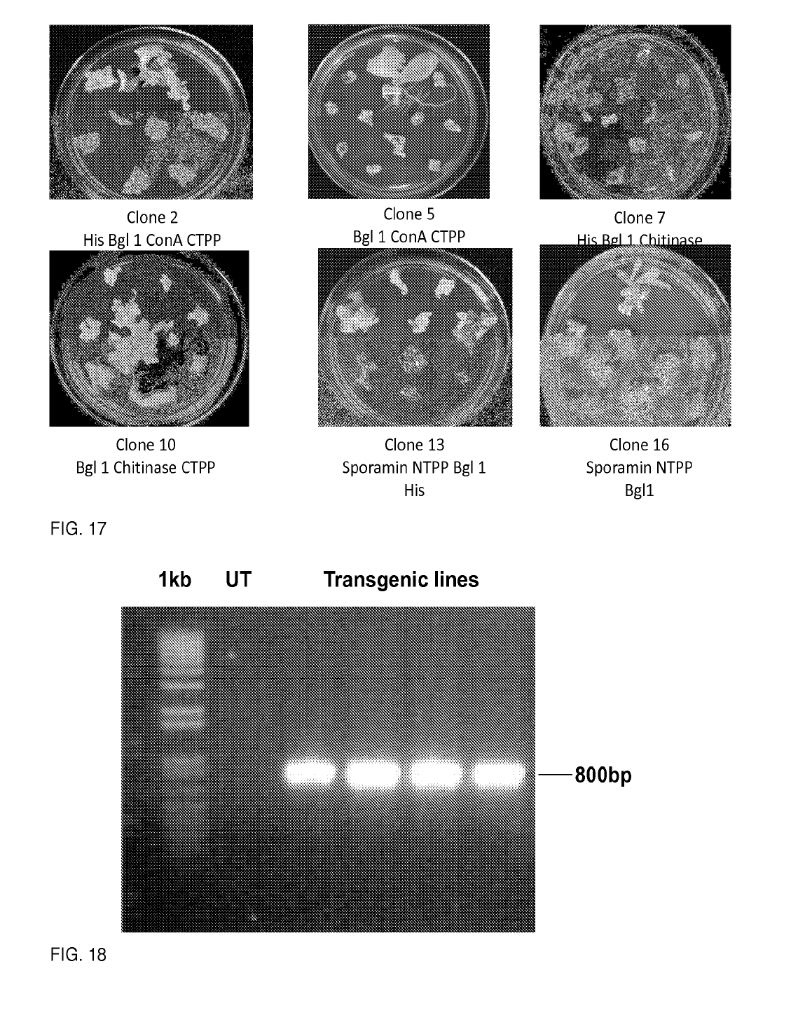

The putative transformant growing on selection medium (FIG. 17) were screened for transgene integration by PCR using gene specific primers. Forward and reverse primers corresponding to the kanamycin resistance selection marker (nptII) were used. Transgenic plants showed amplification of 800 bp fragment as expected (FIG. 18).

Confirmation of Stable Transgene Integration and Copy Number by Southern Blot:

The PCR positive transgenic lines were then selected and screened for presence of transgene and copy number by Southern blot. Southern blot confirmed the Bgl1 gene integration with various copy number. The copy number varied from 1-5 (FIG. 19).

Northern Blot Analysis to Determine Transgene Expression:

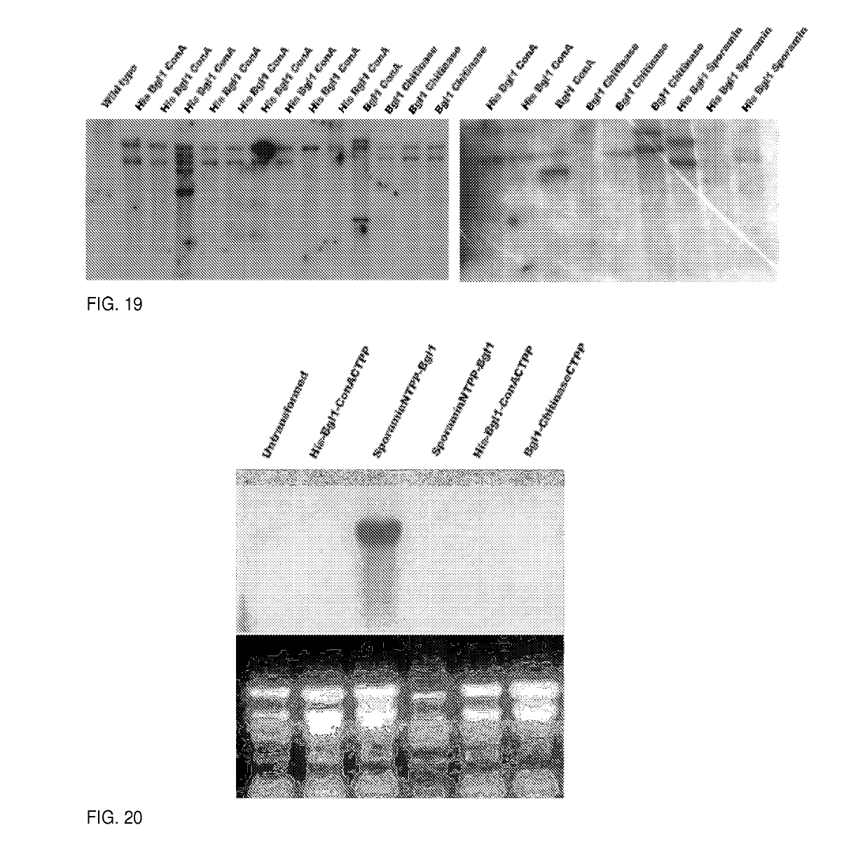

Plants that showed presence of transgene were screened for β-glucosidase transcript by Northern blotting. The membrane containing total RNA was hybridized with radiolabelled β-glucosidase gene specific probe as previously used in Southern blotting. The result showed presence of transcript band in some of the transgenic lines (FIG. 20).

β-Glucosidase Activity:

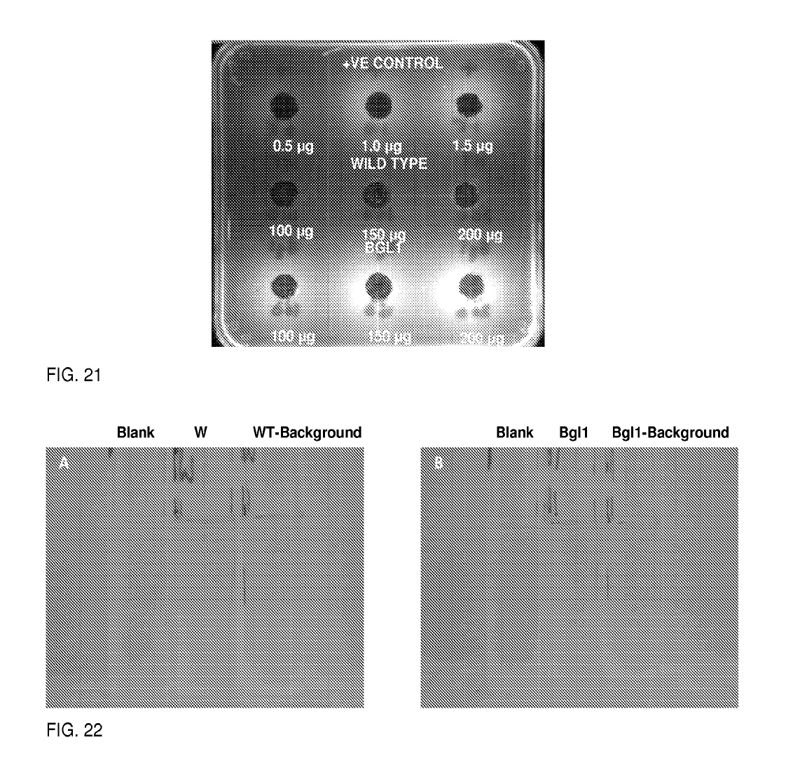

In order to determine enzyme functionality, enzyme assays using substrates pNPG (FIG. 22) and 4-MUG (FIG. 21) were performed. For comparative purposes, transgenic lettuce derived enzyme extracts were incubated in 4-MUG agar plates along with a commercial available glucosidase as a positive control. Both the transgenic and positive control showed halo zones when the plates were illuminated with UV light. The same experimental setup was performed with the positive control and tobacco chloroplast derived enzyme extracts which showed a similar result. Wild type crude extracts were used as negative control.

Optimization of Enzyme Activity Parameters:

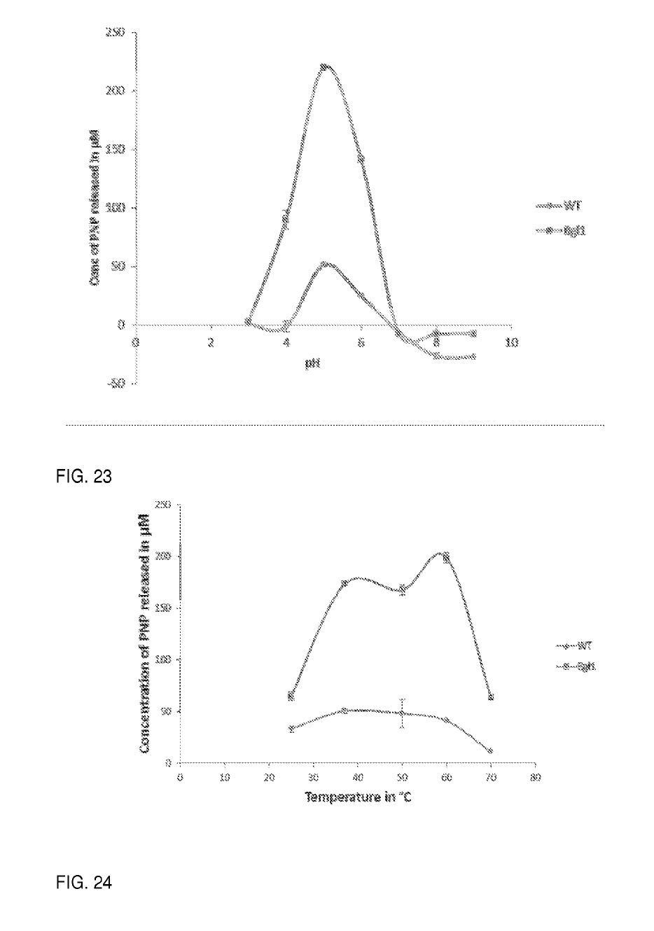

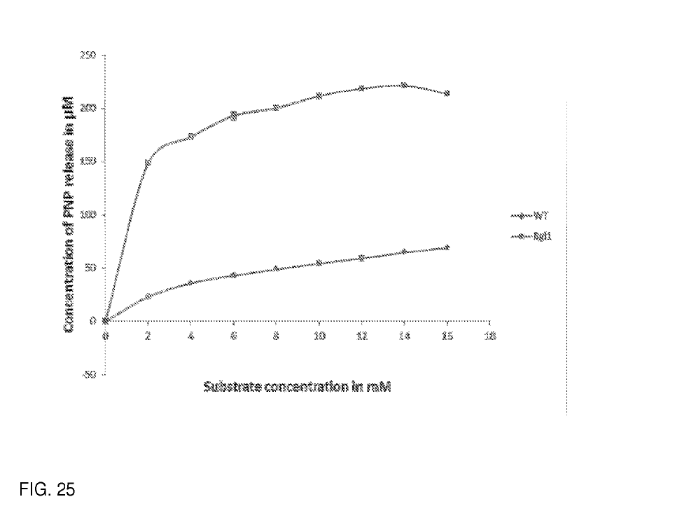

In order to determine the optimum enzyme reaction conditions, the enzyme assay was performed using pNPG substrate at varying pH (FIG. 23), temperature (FIG. 24) and substrate concentrations (FIG. 25). The enzyme activity was seen to be optimum at a pH of 5.0, temperature of 60° C. and at 14 mM substrate concentration. In each setup, the activity of transgenic samples was 4-5 folds in comparison with wild type untransformed samples. In substrate optimization, the enzyme activity increased rapidly at 2 mM concentration and kept increasing upto 14 mM after which the activity started saturating.

### REFERENCES

Aach H, Bode H, Robinson D G, Graebe J E. (1997) ent-Kaurene synthase is located in proplastids of meristematic shoot tissues. Planta 202: 211-219.
Achard P, Genschik P. (2009) Releasing the brakes of plant growth: how GAs shutdown DELLA proteins. J Exp Bot 60: 1085-1092.
An X, Lu J, Huang J D, Zhang B, Liu D, Zhang X, Chen J, Zhou Y, Tong Y. (2007) Rapid assembly of multiple-exon cDNA directly from genomic DNA. PLoS ONE 2: e1179.
Arlen P A, Singleton M, Adamovicz J J, Ding Y, Davoodi-Semiromi A, Daniell H. (2008) Effective plague vaccination via oral delivery of plant cells expressing F1-V antigens in chloroplasts. Infect Immun 76: 3640-3650.
Benková E, Witters E, Van Dongen W, Kolár J, Motyka V, Brzobohatý B, Van Onckelen H A, Machácková I. (1999) Cytokinins in tobacco and wheat chloroplasts: occurrence and changes due to light/dark treatment. Plant Physiol 121: 245-252.
Berghem L E, Pettersson L G. (1974) The mechanism of enzymatic cellulose degradation: isolation and some properties of a beta-glucosidase from Trichoderma viride. Eur J Biochem 46: 295-305.
Brzobohatý B, Moore I, Kristoffersen P, Bako L, Campos N, Schell J, Palme K. (1993) Release of active cytokinin by a beta-glucosidase localized to the maize root meristem. Science 262: 1051-1054.
Carroll A, Somerville C. (2009) Cellulosic biofuels. Annu Rev Plant Biol 60: 165-182.
Curaba J, Moritz T, Blervaque R, Parcy F, Raz V, Herzog M, Vachon G. (2004) AtGA3ox2, a key gene responsible for bioactive gibberellin biosynthesis, is regulated during embryogenesis by LEAFY COTYLEDON2 and FUSCA3 in Arabidopsis. Plant Physiol 136: 3660-3669.
Daniell H, Lee S B, Panchal T, Wiebe P O. (2001) Expression of the native cholera toxin B subunit gene and assembly as functional oligomers in transgenic tobacco chloroplasts. J Mol Biol 311: 1001-1009.
Daniell H, Ruiz O N, Dhingra A. (2004) Chloroplast genetic engineering to improve agronomic traits. Methods Mol Biol 286: 111-137.
Daniell H, Singh N D, Mason H, Streatfield S J. (2009) Plant-made vaccine antigens and biopharmaceuticals. Trends Plant Sci 14: 669-679.
Davies P J. (2004) Plant Hormones: Biosynthesis, Signal Transduction, Action! Kluwer Academic Publishers, Dordrecht, The Netherlands.
Davoodi-Semiromi A, Schreiber M, Nalapalli S, Verma D, Singh N D, Banks R K, Chakrabarti D, Daniell H. (2010) Chloroplast-derived vaccine antigens confer dual immunity against cholera and malaria by oral or injectable delivery. Plant Biotechnol J 8: 223-242.
Esen A. (1993) β-Glucosidases. Esen A, editor., β-Glucosidases: Biochemistry and Molecular Biology. ACS Symposium Series. American Chemical Society, Washington, D.C., pp 1-14.
Garcia-Martinez J L, Ohlrogge J B, Rappaport L. (1981) Differential compartmentation of gibberellin a(₁) and its metabolites in vacuoles of cowpea and barley leaves. Plant Physiol 68: 865-867.
Guo Z, Wagner G J. (1995) Biosynthesis of cembratrienols in cell-free extracts from trichomes of Nicotiana tabacum. Plant Sci 110: 1-10.
Jackson D M, Danehower D A. (1996) Integrated case study: Nicotiana leaf-surface components and their effects on insect pests and diseases. Kerstiens G, editor., Plant Cuticles: An Integrated Functional Approach. BIOS Scientific Publishers, Oxford, pp 231-254.
Jindal V, Dhaliwal G S. (2009) Elucidating resistance in cotton genotypes to whitefly, Bemisia tabaci, by population buildup studies. Phytoparasitica 37: 137-145.
Jordan E T, Hatfield P M, Hondred D, Talón M, Zeevaart J A D, Vierstra R D. (1995) Phytochrome A overexpression in transgenic tobacco. Correlation of dwarf phenotype with high concentrations of phytochrome in vascular tissue and attenuated gibberellin levels. Plant Physiol 107: 797-805.
Kleczkowski K, Schell J. (1995) Phytohormone conjugates: nature and function. Crit Rev Plant Sci 14: 283-298.
Kumar S, Daniell H. (2004) Engineering the chloroplast genome for hyperexpression of human therapeutic proteins and vaccine antigens. Methods Mol Biol 267: 365-383.
Lange T, Kappler J, Fischer A, Frisse A, Padeffke T, Schmidtke S, Lange M J. (2005) Gibberellin biosynthesis in developing pumpkin seedlings. Plant Physiol 139: 213-223.
Lee K H, Piao H L, Kim H Y, Choi S M, Jiang F, Hartung W, Hwang I, Kwak J M, Lee I J, Hwang I. (2006) Activation of glucosidase via stress-induced polymerization rapidly increases active pools of abscisic acid. Cell 126: 1109-1120.
Lee S B, Li B C, Jin S X, Daniell H. (2010) Expression and characterization of antimicrobial peptides Retrocyclin-101 and Protegrin-1 in chloroplasts to control viral and bacterial infections. Plant Biotech J (in press)
Leelavathi S, Gupta N, Maiti S, Ghosh A, Reddy V S. (2003) Overproduction of an alkali- and thermo-stable xylanase in tobacco chloroplasts and efficient recovery of the enzyme. Mol Breed 11: 59-67.
Lin Y, Wagner G J. (1994) Surface disposition and stability of pest-interactive, trichome-exuded diterpenes and sucrose esters of tobacco. J Chem Ecol 20: 1907-1921.
Marion-Poll A, Leung J. (2006) Abscisic acid synthesis, metabolism and signal transduction. In Hedden P, Thomas S G, editors., eds, Plant Hormone Signaling, Annual Plant Reviews, Vol 24 Blackwell Publishing, Oxford, pp 1-36.
McKenzie C L, Weathersbee A A, III, Puterka G J. (2005) Toxicity of sucrose octanoate to egg, nymphal, and adult Bemisia tabaci (Hemiptera: Aleyrodidae) using a novel plant-based bioassay. J Econ Entomol 98: 1242-1247.
Morant A V, Jørgensen K, Jørgensen C, Paquette S M, Sánchez-Pérez R, Møller B L, Bak S. (2008) β-Glucosidases as detonators of plant chemical defense. Phytochemistry 69: 1795-1813.
Nambara E, Marion-Poll A. (2005) Abscisic acid biosynthesis and catabolism. Annu Rev Plant Biol 56: 165-185.
Oliver S N, Dennis E S, Dolferus R. (2007) ABA regulates apoplastic sugar transport and is a potential signal for cold-induced pollen sterility in rice. Plant Cell Physiol 48: 1319-1330.
Perazza D, Vachon G, Herzog M. (1998) Gibberellins promote trichome formation by up-regulating GLABROUS1 in Arabidopsis. Plant Physiol 117: 375-383.
Perlack R D, Wright L L, Turhollow A F, Graham R L, Stokes B J, Erbach D C. (2005) Biomass as Feedstock for a Bioenergy and Bioproducts Industry: The Technical Feasibility of a Billion-Ton Annual Supply. Study Report DOE/GO-102005-2135 (ORNL/TM, -2005/66) Oak Ridge National Laboratory, Oak Ridge, Tenn.
Pimenta Lange M J, Lange T. (2006) Gibberellin biosynthesis and the regulation of plant development. Plant Biol (Stuttg) 8: 281-290.
Rao S K, Prakash A H. (1995) A simple method for the isolation of plant protoplasts. J Biosci 20: 645-655.
Rood S B, Beall F D, Pharis R P. (1986) Photocontrol of gibberellin metabolism in situ in maize. Plant Physiol 80: 448-453.
Rood S B, Pharis R P, Koshioka M. (1983) Reversible conjugation of gibberellins in situ in maize. Plant Physiol 73: 340-346.
Ruhlman T, Verma D, Samson N, Daniell H. (2010) The role of heterologous chloroplast sequence elements in transgene integration and expression. Plant Physiol 152: 2088-2104.
Salas Fernandez M G, Becraft P W, Yin Y, Lübberstedt T. (2009) From dwarves to giants? Plant height manipulation for biomass yield. Trends Plant Sci 14: 454-461.
Sambrook J, Russell D W. (2001). Molecular Cloning: A Laboratory Manual, Ed 3 Cold Spring Harbor Laboratory Press, Cold Spring Harbor, N.Y.
Schliemann W. (1984) Hydrolysis of conjugated gibberellins by β-glucosidases of dwarf rice (Oryza sativa L. cv. ‘Tan ginbozu’). J Plant Physiol 116: 123-132.
Schliemann W, Schneider G. (1979) Untersuchungen zur enzymatischen: Hydrolyse von Gibberellin-O-glucosiden. I. Hydrolysegeschwindigkeiten von Gibberellin-13-Oglucosiden. Biochem Physiol Pflanzen 174: 738-745.
Schneider G, Jensen E, Spray C R, Phinney B O. (1992) Hydrolysis and reconjugation of gibberellin A20 glucosyl ester by seedlings of Zea mays L. Proc Natl Acad Sci USA 89: 8045-8048.
Schneider G, Koch M, Fuchs P, Schmidt J. (2000) Identification of metabolically formed glucosyl conjugates of [17-D2] GA34. Phytochem Anal 11: 232-235.
Schneider G, Schliemann W. (1994) Gibberellin conjugates: an overview. Plant Growth Regul 15: 247-260.
Schreiber K, Weiland J, Sembdner G. (1970) Isolierung von Gibberellin-A₈-O(3)-β-d-glucopyranosid aus Frtüchten von Phaseolus coccineus. Phytochemistry 9: 189-198.
Schwab B, Folkers U, Ilgenfritz H, Hülskamp M. (2000) Trichome morphogenesis in Arabidopsis. Philos Trans R Soc Lond B Biol Sci 355: 879-883.
Sembdner G, Atzorn R, Schneider G. (1994) Plant hormone conjugation. Plant Mol Biol 26: 1459-1481.
Sembdner G, Grofl D, Liebisch H W, Schneider G. (1980) Biosynthesis and metabolism of plant hormones. MacMillan J, editor., Encyclopedia of Plant Physiology. Springer-Verlag, Berlin, pp 281-444.
Severson R F, Arrendale R F, Chortyk O T, Johnson A W, Jackson D M, Gwynn G R, Chaplin J F, Stephenson M G. (1984) Quantitation of the major cuticular components from green leaf of different tobacco types. J Agric Food Chem 32: 566-570.
Sticklen M B. (2008) Plant genetic engineering for biofuel production: towards affordable cellulosic ethanol. Nat Rev Genet 9: 433-443.
Taylor L E, II, Dai Z, Decker S R, Brunecky R, Adney W S, Ding S Y, Himmel M E. (2008) Heterologous expression of glycosyl hydrolases in planta: a new departure for biofuels. Trends Biotechnol 26: 413-424.
Verma D, Daniell H. (2007) Chloroplast vector systems for biotechnology applications. Plant Physiol 145: 1129-1143.
Verma D, Kanagaraj A, Jin S X, Singh N D, Kolattukudy P E, Daniell H. (2010a) Chloroplast-derived enzyme cocktails hydrolyse lignocellulosic biomass and release fermentable sugars. Plant Biotechnol J 8: 332-350.
Verma D, Moghimi B, LoDuca P A, Singh H D, Hoffman B E, Herzog R W, Daniell H. (2010b) Oral delivery of bioencapsulated coagulation factor IX prevents inhibitor formation and fatal anaphylaxis in hemophilia B mice. Proc Natl Acad Sci USA 107: 7101-7106.
Verma D, Samson N P, Koya V, Daniell H. (2008) A protocol for expression of foreign genes in chloroplasts. Nat Protoc 3: 739-758.
Wagner G J, Wang E, Shepherd R W. (2004) New approaches for studying and exploiting an old protuberance, the plant trichome. Ann Bot (Lond) 93: 3-11.
Wang E, Wang R, DeParasis J, Loughrin J H, Gan S, Wagner G J. (2001) Suppression of a P450 hydroxylase gene in plant trichome glands enhances natural-product-based aphid resistance. Nat Biotechnol 19: 371-374.
Wei S, Marton I, Dekel M, Shalitin D, Lewinsohn E, Bravdo B A, Shoseyov O. (2004) Manipulating volatile emission in tobacco leaves by expressing Aspergillus niger beta-glucosidase in different subcellular compartments. Plant Biotechnol J 2: 341-350.
Wiese G, Grambow H J. (1986) Indole-3-methanol-β-d-glucoside and indole-3-carboxylic acid-β-glucoside are products of indole-3-acetic acid degradation in wheat leaf segments. Phytochemistry 25: 2451-2455.
Woodward A W, Bartel B. (2005) Auxin: regulation, action, and interaction. Ann Bot (Lond) 95: 707-735.
Yamaguchi S, Kamiya Y, Sun T P. (2001) Distinct cell-specific expression patterns of early and late gibberellin biosynthetic genes during Arabidopsis seed germination. Plant J 28: 443-453.
Yao J, Pang Y, Qi H, Wan B, Zhao X, Kong W, Sun X, Tang K. (2003) Transgenic tobacco expressing Pinellia ternata agglutinin confers enhanced resistance to aphids. Transgenic Res 12: 715-722.
Yu L X, Gray B N, Rutzke C J, Walker L P, Wilson D B, Hanson M R. (2007) Expression of thermostable microbial cellulases in the chloroplasts of nicotine-free tobacco. J Biotechnol 131: 362-369.
Zhang X F, Zhang S F, Shi X Y, Li J, Yang R L. (2004) Acute LD₅₀ determination of toosendanin in mice by ip injection. J Traditional Chinese Veterinary Med 3: 12-13.
Ziegelhoffer T, Raasch J A, Austin-Phillips S. (2009) Expression of Acidothermus cellulolyticus E1 endo-beta-1,4-glucanase catalytic domain in transplastomic tobacco. Plant Biotechnol J 7: 527-536.

While various disclosed embodiments have been described above, it should be understood that they have been presented by way of example only, and not limitation. Numerous changes to the subject matter disclosed herein can be made in accordance with this Disclosure without departing from the spirit or scope of this Disclosure. In addition, while a particular feature may have been disclosed with respect to only one of several implementations, such feature may be combined with one or more other features of the other implementations as may be desired and advantageous for any given or particular application.

Thus, the breadth and scope of the subject matter provided in this Disclosure should not be limited by any of the above explicitly described embodiments. Rather, the scope of this Disclosure should be defined in accordance with the following claims and their equivalents.

The terminology used herein is for the purpose of describing particular embodiments only and is not intended to be limiting. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art to which embodiments belong. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The teachings of any patents, patent applications, technical or scientific articles or other references are incorporated herein in their entirety to the extent not inconsistent with the teachings herein.

TABLE I
Phenotypic assessment of transplastomic BGL-1 and UT plants

| | | | | | | Density of | |
|---|---|---|---|---|---|---|---|
| | | Plant | Leaf Area ± | | | Trichomes ± sd | |
| | Internode | Height ± | sd^{a} | Flowering | Biomass ± | Upper | Lower |
| Plant | Length ± sd | sd | cm² per | Time ± sd | sd^{b} | Side | Side |
| Type | cm | | leaf | d | g per plant | mm² | |
| UT | 3.2 ± 0.3 | 135.3 ± 5 | 492.2 ± 116 | 152 ± 6 | 584.2 ± 33 | 14.5 ± 3 | 21.6 ± 4 |
| BGL-1 | 5.8 ± 0.4 | 204.3 ± 12 | 779.4 ± 113 | 120 ± 5 | 1,104.3 ± 55 | 149.9 ± 10 | 160.6 ± 5 |

^{a}A total of 24 mature leaves (counted from the top, the seventh, and eighth) from 12 BGL-1 and 12 UT plants were measured with the 3,100 leaf area meter.
^{b}Five fully grown plants (with 14-16 leaves) from BGL-1 and UT were weighed after removal of soil. The whole plants (including the leaves, stems, and roots) were weighed on a Metter electronic bal

TABLE II
BGL-1 enzyme yield in transplastomic tobacco plants
One unit of BGL-1 enzyme is defined as the amount of enzyme that released 1 μmol of p-
nitrophenol from pNPG substrate under the assay conditions described in “Materials and
Methods.” Enzyme yield per acre per year was determined using the following information:
1,5941.4 BGL-1 units per plant × 8,000 plants per acre × three cuttings per year = 382.59 million
units per acre per year.

| | | No. of | Average | | | | Whole | Units | Units |
|---|---|---|---|---|---|---|---|---|---|
| | | Leaves | Weight | Units | Units | | Plant | (Millions) | (Millions) |
| | Leaf | per | per Leaf | g⁻¹ in | Per | Per Age | Yield | per Acre | per Acre |
| Enzyme | Age | Plant | g | Leaf | Leaf | Group | units | per Cutting | per Year |
| BGL-1 | Young | 5.7 | 7.1 | 9.24 | 65.6 | 373.92 | 1,5941.4 | 127.53 | 382.59 |
| | Mature | 14.2 | 16.5 | 44.41 | 732.77 | 10,405.33 | | | |
| | Old | 8.2 | 14.9 | 42.25 | 629.53 | 5,126.15 | | | |

TABLE III
Quantification of endogenous GAs from different parts of UT and
BGL-1 transplastomic lines Results are means of two determinations.

| | Mature | | | | | | | | | |
|---|---|---|---|---|---|---|---|---|---|---|
| | Leaf | | Inflorescence | | Shoot Tip | | Internode | | Young Leaf | |
| | UT | BGL-1 | UT | BGL-1 | UT | BGL-1 | UT | BGL-1 | UT | BGL-1 |
| GA | ng g²¹dry wt | | | | | | | | | |
| GA₅₃ | 0.3 | 0.2 | 2.8 | 3.1 | 0.9 | 1.0 | 5.0 | 7.0 | 0.2 | 0.4 |
| GA₄₄ | 0.0 | 0.1 | 0.0 | 0.3 | 0.3 | 0.7 | 0.7 | 3.9 | 0.1 | 0.3 |
| GA₁₉ | 5.7 | 10.6 | 12.6 | 11.2 | 14.3 | 20.8 | 18.7 | 23.9 | 13.0 | 23.8 |
| GA₂₀ | 2.4 | 7.6 | 0.5 | 0.6 | 5.9 | 8.1 | 3.9 | 4.8 | 19.4 | 36.8 |
| GA₁ | 3.3 | 6.7 | 0.9 | 0.3 | 1.9 | 1.5 | 1.4 | 0.4 | 10.0 | 15.6 |
| GA₈ | 1.5 | 2.9 | 1.0 | 0.7 | 2.1 | 0.8 | 0.7 | 0.7 | 0.8 | 0.7 |
| GA₄ | 0.4 | 0.6 | 0.3 | 0.7 | 0.4 | 0.6 | 1.0 | 0.2 | 2.3 | 6.6 |
| GA₃₄ | 0.0 | 0.1 | 0.1 | 0.0 | 0.0 | 0.1 | 0.0 | 0.0 | 0.1 | 0.2 |

TABLE IV
Division and plating efficiency of protoplasts derived from BGL-1 and UT

| | | | | | | |
|---|---|---|---|---|---|---|
| Hormone | No. of | | Division | No. of | | Plating |
| Combination | Protoplasts | No. of | Efficiency^{a} | Protoplasts | No. of | Efficiency^{b} |
| (mg L⁻¹) | Observed | Divisions | % | Observed | Calli | % |

Free hormone

| | | | | | | |
|---|---|---|---|---|---|---|
| UT | 382 | 0 | 0 | 532 | 0 | 0 |
| | 428 | 0 | | 432 | 0 | |
| | 469 | 0 | | 470 | 0 | |
| BGL-1 | 482 | 17 | 2.8 ± 0.7 | 411 | 9 | 2.0 ± 0.3 |
| | 478 | 13 | | 485 | 11 | |
| | 467 | 10 | | 421 | 7 | |

Zeatin (1.0) + naphthylacetic acid (3.0)

| | | | | | | |
|---|---|---|---|---|---|---|
| UT | 510 | 134 | 28.0 ± 1.7 | 531 | 51 | 9.1 ± 0.6 |
| | 514 | 145 | | 504 | 47 | |
| | 481 | 142 | | 469 | 40 | |
| BGL-1 | 412 | 141 | 34.0 ± 2.0 | 410 | 62 | 14.1 ± 1.4 |
| | 514 | 185 | | 519 | 77 | |
| | 489 | 156 | | 481 | 60 | |

Naphthylacetic acid (3.0)

| | | | | | | |
|---|---|---|---|---|---|---|
| UT | 397 | 22 | 4.8 ± 0.7 | 389 | 12 | 3.3 ± 0.2 |
| | 413 | 20 | | 421 | 14 | |
| | 419 | 17 | | 456 | 16 | |
| BGL-1 | 567 | 41 | 9.1 ± 1.8 | 512 | 25 | 4.3 ± 0.5 |
| | 418 | 45 | | 504 | 20 | |
| | 421 | 39 | | 462 | 19 | |

Naphthylacetic acid (3.0) + zeatin-O-glucoside (1.0)

| | | | | | | |
|---|---|---|---|---|---|---|
| UT | 501 | 18 | 4.1 + 0.5 | 512 | 18 | 3.4 + 0.4 |
| | 402 | 19 | | 461 | 17 | |
| | 479 | 20 | | 470 | 14 | |
| BGL-1 | 510 | 132 | 27.5 ± 2.2 | 439 | 52 | 11.4 ± 1.0 |
| | 408 | 122 | | 481 | 59 | |
| | 412 | 110 | | 477 | 49 | |

Zeatin (1.0)

| | | | | | | |
|---|---|---|---|---|---|---|
| UT | 512 | 35 | 6.8 ± 0.9 | 514 | 23 | 4.6 ± 0.3 |
| | 427 | 32 | | 525 | 26 | |
| | 501 | 30 | | 426 | 19 | |
| BGL-1 | 433 | 55 | 10.6 ± 0.8 | 421 | 28 | 6.2 ± 0.4 |
| | 479 | 49 | | 513 | 31 | |
| | 534 | 48 | | 401 | 24 | |

Zeatin-O-glucoside (1.0)

| | | | | | | |
|---|---|---|---|---|---|---|
| UT | 521 | 0 | 0 | 458 | 0 | 0 |
| | 520 | 0 | | 421 | 0 | |
| | 524 | 0 | | 521 | 0 | |
| BGL-1 | 551 | 36 | 7.5 ± 1.2 | 471 | 18 | 3.7 ± 0.3 |
| | 478 | 42 | | 440 | 15 | |
| | 499 | 37 | | 502 | 20 | |

^{a}Number of protoplasts dividing per number of total protoplasts in the same visual field of the microscope.
^{b}Number of protoplasts dividing and forming cell groups per number of total protoplasts in the same visual field of the microscope.

TABLE V
Aphid and whitefly colonization tests on BGL-1 and UT plants
Whole plants (40 d old, six- to seven-leaf stage) were confined to insect-proof nylon mesh bags
and maintained at 25° C. for 25 d as shown in FIG. 7. For the aphid bioassay, 30 neonatal
nymphs were introduced with a hair brush to each plant. Thirty newly emerged adult whiteflies
were released in each mesh bag. Twenty-five days after release, the mesh bags were removed,
and the total number of adults and immature-stage insects that emerged was recorded on the
whole plant. Both BGL-1 and UT lines for aphid or whitefly colonization tests had 10 plants
each. Values shown are numbers per plant ± sd.

| | Whitefly Population | | | Aphid Population | | |
|---|---|---|---|---|---|---|
| Plant Type | Eggs/Pupae | Adults | Total | Nymphs | Adults | Total |
| UT | 1,257.8 ± 171 | 580.6 ± 71 | 1,838.4 ± 222 | 568.9 ± 101 | 388.9 ± 61 | 957.8 ± 156 |
| BGL-1 | 75.0 ± 15 | 25.6 ± 5 | 100.6 ± 17 | 42.0 ± 13 | 22.3 ± 5 | 64.3 ± 15 |

TABLE 6
Primers to amplify Bgl1 including vacuole targeting sequences with or without his tag

| No. | Primer | Sequence |
|---|---|---|
| DV1 | Bgl1 For | 5′-AAC CgA ATT CTA CgT ACA TAT gCg TTA CCg AAC AgC AgC-3′ |
| DV2 | ConA CTPP Bgl1 Rev1 | 5′-gTA gCA ATg TCC ggg ATC TCC gCT ACC gAC AgA gTg CTC gTC-3′ |
| DV3 | ConA CTPP Bgl1 Rev2 | 5′-AAT CCC CCg ggT CTA gAC TAA ACC ACg gTA gCA ATg TCC ggg ATC |
| | | TCC gCT ACC-3′ |
| DV4 | Chitinase CTPP Bgl1 Rev1 | 5′-gTA TCg ACT AAA AgT CCg TTT CCC gCT ACC gAC AgA gTg CTC gTC-3′ |
| DV5 | Chitinase CTPP Bgl1 Rev2 | 5′-ggA ATC CCC Cgg gTC TAg ACT ACA TAg TAT CgA CTA AAA gTC CgT |
| | | TTC CCg CTA CCg-3′ |
| DV6 | 5′ His Bgl1 For | 5′-AAC CgA ATT CTA CgT ACA TAT gCA CCA CCA CCA CCA CCA CAT gCg |
| | | TTA CCg AAC AgC AgC-3′ |
| DV7 | Sporamin NTPP Bgl1 | 5′-ATC CCA TCC gCC TCC CCA CCA CAC ACg AAC CCg CCA TgC gTT ACC |
| | For1 | gAA CAg CAg CT-3′ |
| DV8 | Sporamin NTPP Bgl1 | 5′-CCg AAT TCT ACg TAC ATA TgC ATT CCA ggT TCA ATC CCA TCC gCC |
| | For2 | TCC CCA CCA CAC AC-3′ |
| DV9 | Bgl1 Rev | CTC TCT AgA CTA CgC TAC CgA CAg AgT gCT CgT C |
| DV10 | 3′ His Bgl1 Rev | 5′-AAT CCC CCg ggT CTA gAC TAg Tgg Tgg Tgg Tgg Tgg TgC gCT ACC |
| | | gAC AgA gTg CTC gTC-3′ |

TACgTA - SnaBI restriction site,
TCTAgA - XbaI restriction site

What is claimed is:

1. A method of producing a transgenic plant with Bgl overexpression relative to a wild-type plant, said method comprising: (a) introducing into a plant cell an expression cassette that comprises a Bgl gene to thereby produce a transformed plant cell; and (b) producing a transgenic plant from the transformed plant cell, wherein the transgenic plant has increased biomass, increased height, increased trichome density or increased seed production relative to a wild type plant.

2. The method of claim 1, wherein the Bgl gene is operably linked to a promoter selected from the group consisting of a tissue-specific promoter, an inducible promoter and a constitutive promoter.

3. The method of claim 1, wherein the Bgl gene is Bgl1.

4. The method of claim 1, wherein said Bgl gene is linked with a vacuole targeting sequence.

5. The method of claim 1, wherein said expression cassette is introduced into a plastid of said plant cell.

6. The method of claim 5, wherein said expression cassette comprises, as operably linked components, a promoter operative in said plastid, a selectable marker sequence, a heterologous polynucleotide sequence coding for a Bgl gene, transcription termination functional in said plastid, and flanking each side of the expression cassette, flanking DNA sequences which are homologous to a DNA sequence of a plastid genome of said plastid, whereby stable integration of the heterologous coding sequence into the plastid genome of the target plant is facilitated through homologous recombination of the flanking sequence with the homologous sequences in said plastid genome.

7. The method of claim 5, wherein the plastid is selected from the group consisting of chloroplasts, chromoplasts, amyloplasts, proplastide, leucoplasts and etioplasts.

8. The method of claim 6, wherein the selectable marker sequence is an antibiotic-free selectable marker.

9. A transgenic plant that overexpresses Bgl1 relative to a corresponding wild-type plant, wherein said transgenic plant has increased biomass, increased height, increased trichome density or increased seed production relative to a wild type plant.

10. The transgenic plant of claim 9, which comprises a plastid stably transformed with a plastid transformation vector that comprises an expression cassette comprising, as operably linked components in the 5′ to the 3′ direction of translation, a promoter operative in said plastid, a selectable marker sequence, a heterologous polynucleotide sequence coding for Bgl gene, transcription termination functional in said plastid, and flanking each side of the expression cassette, flanking DNA sequences which are homologous to a DNA sequence of a plastid genome of said plastid, whereby stable integration of the heterologous coding sequence into the plastid genome of the target plant is facilitated through homologous recombination of the flanking sequence with the homologous sequences in said plastid genome.

11. The transgenic plant of claim 9 which is a monocotyledonous or dicotyledonous plant.

12. The transgenic plant of claim 9 which is maize, rice, grass, rye, barley, oat, wheat, soybean, peanut, grape, potato, sweet potato, pea, canola, tobacco, tomato or cotton.

13. The transgenic plant of claim 9 which is edible for mammals and humans.

14. The transgenic plant of claim 9, which is a plant selected from the group consisting of acacia, alfalfa, apple, apricot, artichoke, ash tree, asparagus, avocado, banana, barley, beans, beet, birch, beech, blackberry, blueberry, broccoli, brussels sprouts, cabbage, canola, cantaloupe, carrot, cassava, cauliflower, cedar, a cereal, celery, chestnut, cherry, Chinese cabbage, citrus, Clementine, clover, coffee, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, okra, onion, orange, an ornamental plant or flower or tree, papaya, palm, parsley, parsnip, pea, peach, peanut, pear, peat, pepper, persimmon, pigeon pea, pine, pineapple, plantain, plum, pomegranate, potato, pumpkin, radicchio, radish, rapeseed, raspberry, rice, rye, sorghum, sallow, spinach, spruce, squash, strawberry, sugarbeet, sugarcane, sunflower, sweet potato, sweet corn, tangerine, tea, tobacco, tomato, trees, triticale, turf grasses, turnips, a vine, walnut, watercress, watermelon, wheat, yams, yew, and zucchini.

15. The transgenic plant of claim 9, wherein said transgenic plant is Cannabis sativa, Papaver somniferum or Erythorxylum coca.

16. The transgenic plant of claim 9, wherein said transgenic plant comprises a plant cell transformed with an expression cassette that comprises a Bgl gene linked with a vacuole targeting sequence encoding a vacuole targeting peptide.

17. The transgenic plant of claim 16, wherein said vacuole targeting peptide is a C-terminal propeptide (CTPP) of Concanavalin A, a Chitinase A and/or N-terminal propeptide (NTPP) or sporamin.

18-25. (canceled)

26. A method of releasing native phytohormones associated with a plant cell, said method comprising engineering said plant cell so as to express heterologous Bgl1, wherein expression of heterologous Bgl1 increases B-glucosidase activity in said cell which releases native phytohormones in said plant cell.

27. The method of claim 26, wherein said native phytohormones are in a conjugated state prior to being exposed to B-glucosidase expressed in said plant cell.

28-31. (canceled)


SEQUENCE LISTING (19 sequences)

SEQ ID NO: 1 - DNA, 39 bp, Artificial Sequence
  Synthetic Primer
aaccgaattctacgtacatatgcgttaccgaacagcagc

SEQ ID NO: 2 - DNA, 42 bp, Artificial Sequence
  Synthetic Primer
gtagcaatgtccgggatctccgctaccgacagagtgctcgtc

SEQ ID NO: 3 - DNA, 54 bp, Artificial Sequence
  Synthetic Primer
aatcccccgggtctagactaaaccacggtagcaatgtccgggatctccgctacc

SEQ ID NO: 4 - DNA, 45 bp, Artificial Sequence
  Synthetic Primer
gtatcgactaaaagtccgtttcccgctaccgacagagtgctcgtc

SEQ ID NO: 5 - DNA, 57 bp, Artificial Sequence
  Synthetic Primer
ggaatcccccgggtctagactacatagtatcgactaaaagtccgtttcccgctaccg

SEQ ID NO: 6 - DNA, 60 bp, Artificial Sequence
  Synthetic Primer
aaccgaattctacgtacatatgcaccaccaccaccaccacatgcgttaccgaacagcagc

SEQ ID NO: 7 - DNA, 56 bp, Artificial Sequence
  Synthetic Primer
atcccatccgcctccccaccacacacgaacccgccatgcgttaccgaacagcagct

SEQ ID NO: 8 - DNA, 59 bp, Artificial Sequence
  Synthetic Primer
ccgaattctacgtacatatgcattccaggttcaatcccatccgcctccccaccacacac

SEQ ID NO: 9 - DNA, 34 bp, Artificial Sequence
  Synthetic Primer
ctctctagactacgctaccgacagagtgctcgtc

SEQ ID NO: 10 - DNA, 60 bp, Artificial Sequence
  Synthetic Primer
aatcccccgggtctagactagtggtggtggtggtggtgcgctaccgacagagtgctcgtc

SEQ ID NO: 11 - DNA, 90 bp, Artificial Sequence
  Synthetic Primer
gaattccatatgcgttaccgaacagcagctgcgctggcacttgccactgggccctttgctagggcagacagtcactcaacatcgggggcc

SEQ ID NO: 12 - DNA, 39 bp, Artificial Sequence
  Synthetic Primer
ctagggcagacagtcactcaacatcgggggcctcggctg

SEQ ID NO: 13 - DNA, 59 bp, Artificial Sequence
  Synthetic Primer
cacgccgcggtacgagttcggctatggactgtcttacaccaagttcaactactcacgcc

SEQ ID NO: 14 - DNA, 25 bp, Artificial Sequence
  Synthetic Primer
acagtccatagccgaactcgtaccg

SEQ ID NO: 15 - DNA, 34 bp, Artificial Sequence; identical to SEQ ID NO: 9
  Synthetic Primer

SEQ ID NO: 16 - DNA, 25 bp, Artificial Sequence
  Synthetic Primer
aaaacccgtcctcagttcggattgc

SEQ ID NO: 17 - DNA, 25 bp, Artificial Sequence
  Synthetic Primer
ccgcgttgtttcatcaagccttacg

SEQ ID NO: 18 - DNA, 24 bp, Artificial Sequence
  Synthetic Primer
ctgtagaagtcaccattgttgtgc

SEQ ID NO: 19 - DNA, 24 bp, Artificial Sequence
  Synthetic Primer
tgactgcccacctgagagcggaca